(19) United States
(12) Patent Application Publication
(10) Pub. No.: US 2025/0188038 A1
(43) Pub. Date: Jun. 12, 2025

(54) CRYSTALLINE FORMS OF N-{[(S)-{[3-(4-CHLOROPHENYL)-4-PHENYL-4,5-DIHYDRO-1H-PYRAZOL-1-YL][4-(TRIFLUOROMETHYL)BENZENE

(71) Applicant: Novo Nordisk A/S, Bagsvaerd (DK)

(72) Inventors: Derik McCarthy, Hillsborough, NJ (US); Mitulkumar Patel, Hillsborough, NJ (US); Jun Zhou, Hubei (CN); Cheng Cheng, Hubei (CN)

(21) Appl. No.: 18/836,231

(22) PCT Filed: Feb. 8, 2023
(86) PCT No.: PCT/CA2023/050163

§ 371 Date: Aug. 6, 2024

Related U.S. Application Data
(60) Provisional application No. 63/267,734, filed on Feb. 9, 2022.

(51) Int. Cl. C07D 231/06; A61K 31/415

(52) U.S. Cl. CPC C07D 231/06; A61K 31/415

(57) ABSTRACT

Crystalline Forms of a compound of Formula I are provided: (I) Crystalline Form C is among the crystalline Forms identified. Form C has an X-ray powder diffraction (XRPD) pattern having characteristic peaks expressed in degrees 2θ (±0.2° 2θ) at 6.46, 15.88, 19.44 and 5.86, and a Differential Scanning Calorimetry (DSC) thermogram that exhibits an endotherm having an onset at 157.1° C.

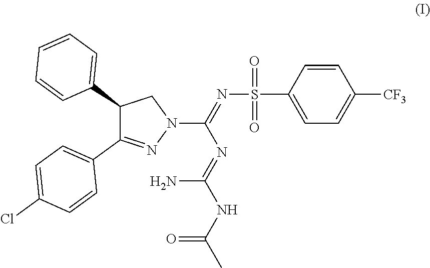 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

### TECHNICAL FIELD

The technical field relates to crystalline forms of the compound N-N′-((S)-3-(4-chlorophenyl)-4-phenyl-4,5-dihydro-1H-pyrazol-1-yl)(((4-(trifluoromethyl)phenyl) sulfonyl)imino)methyl) carbamimidoyl) acetamide, as well as pharmaceutical compositions, therapeutic uses thereof and processes of manufacture.

### BACKGROUND

It is generally known that activation of the cannabinoid CB₁ receptor increases appetite, increases the biosynthesis and storage of lipids, inhibits the actions of insulin and leptin, and promotes inflammation and fibrosis. Research was thus focused on developing CB₁ receptor inhibitors for the potential treatment of obesity and the metabolic disorder associated therewith, referred to as metabolic syndrome. Rimonabant was shown effective in treating metabolic syndrome but caused neuropsychiatric (i.e. CNS-related) side effects, which resulted in its withdrawal from the market.

Compounds preferentially targeting the CB₁ receptor in peripheral tissues (e.g. adipose tissue, liver, muscle, lung, kidney, macrophages, pancreatic beta cells and gastrointestinal tract), while not interacting with CB₁ receptors in brain tissue, thereby avoiding or reducing CNS-related side effects, were disclosed by George Kunos et al. in U.S. Pat. No. 9,765,031.

One of the compounds is N-{[(S)-{[3-(4-chlorophenyl)-4-phenyl-4,5-dihydro-1H-pyrazol-1-yl][4-(trifluoromethyl)benzenesulfonamido]methylidene}amino]methanimidoyl}acetamide, for which it is desirable to identify stable crystalline forms, that may be suitable for therapeutic use.

### SUMMARY

In one aspect, there is provided a compound of Formula I:

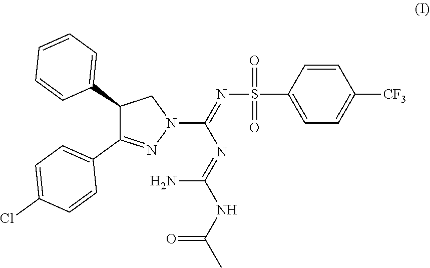 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and exhibits an X-ray powder diffraction (XRPD) pattern having characteristic peaks expressed in degrees 2θ (±0.2° 2θ) at 6.46, 15.88, 19.44 and 5.86.

In some embodiments, the XRPD pattern further has characteristic peaks expressed in degrees 2θ (±0.2° 2θ) at 7.46, 22.15 and 26.24.

In some embodiments, the XRPD pattern further has characteristic peaks expressed in degrees 2θ (±0.2° 2θ) at 9.85, 19.07, 22.77.

In some embodiments, the XRPD pattern further has characteristic peaks expressed in degrees 2θ (±0.2° 2θ) at 3.81, 17.17 and 20.84.

In some embodiments, the XRPD pattern further has characteristic peaks expressed in degrees 2θ (±0.2° 2θ) at 17.95, 15.43 and 24.24.

In another aspect, there is provided a compound of Formula I:

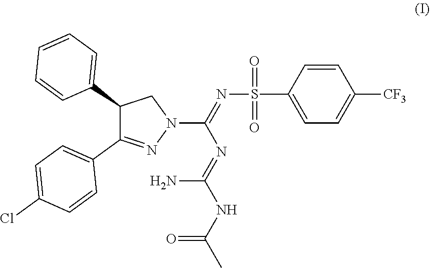 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and has a Differential Scanning calorimetry (DSC) thermogram that exhibits an endotherm having an onset of about 157.1° C.

In another aspect, there is provided a compound of Formula I:

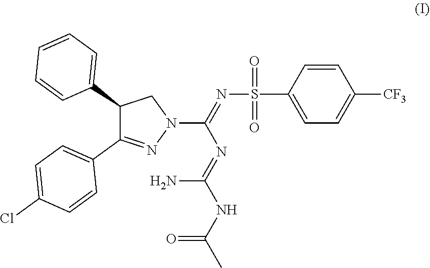 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and has a Differential Scanning calorimetry (DSC) thermogram that exhibits an endotherm having a peak temperature of about 167.9° C.

In another aspect, there is provided a compound of Formula I:

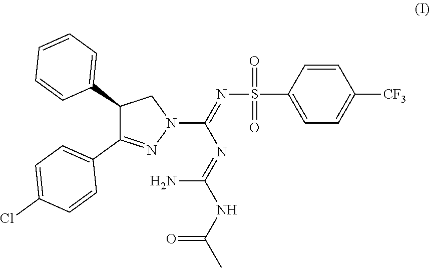 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which has an X-ray powder diffraction pattern substantially the same as shown in FIG. 4A-Form C.

In another aspect, there is provided a compound of Formula I:

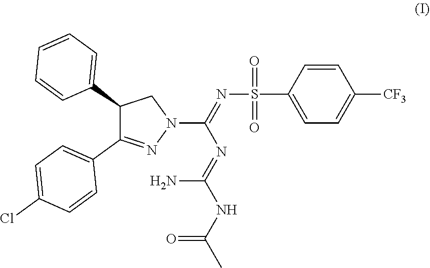 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and exhibits an X-ray powder diffraction (XRPD) pattern having characteristic peaks expressed in degrees 2θ (±0.2° 2θ) at 5.89 and 17.39.

In some embodiments, the XRPD pattern further has characteristic peaks expressed in degrees 2θ (±0.2° 2θ) at 9.39 and 17.80.

In some embodiments, the XRPD pattern further has a characteristic peak expressed in degrees 2θ (±0.2° 2θ) at 12.96.

In some embodiments, the XRPD pattern further has a characteristic peak expressed in degrees 2θ (±0.2° 2θ) at 19.85.

In some embodiments, the XRPD pattern further has a characteristic peak expressed in degrees 2θ (±0.2° 2θ) at 16.96.

In another aspect, there is provided a compound of Formula I:

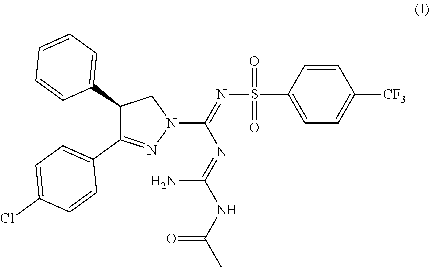 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and has a Differential Scanning calorimetry (DSC) thermogram that exhibits an endotherm having an onset of about 152.2° C.

In another aspect, there is provided a compound of Formula I:

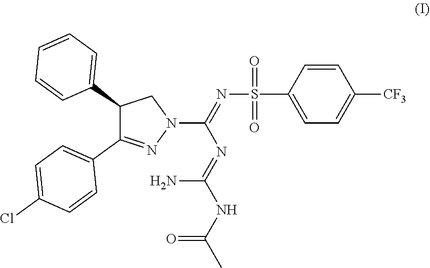 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which is crystalline and has a Differential Scanning calorimetry (DSC) thermogram that exhibits an endotherm having a peak temperature of about 162.3° C.

In another aspect, there is provided a compound of Formula I:

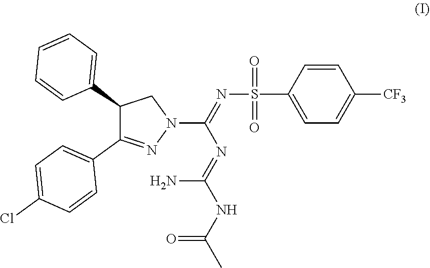 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

which has an X-ray powder diffraction pattern substantially the same as shown in FIG. 2A.

In some embodiments, the compound of Formula I comprises one crystalline form at a purity of 95% or higher.

In some embodiments, the purity is of 99% or higher.

In some embodiments, the purity is of 99.8% or higher.

In some embodiments, the compound of Formula I is substantially pure.

In another aspect, there is provided a pharmaceutical composition, comprising the compound of Formula I as described herein, and a pharmaceutically acceptable carrier or excipient.

In some embodiments, the pharmaceutical composition is formulated as an oral dosage form.

In some embodiments, the oral dosage form is a tablet, a capsule, a lozenge, a pastille or a granule.

In some embodiments, the pharmaceutical composition is formulated as an oral suspension.

In another aspect, there is provided the use of the compound of Formula I as described herein or the pharmaceutical composition as described herein, for the treatment of a disease or disorder selected from the group consisting of: obesity (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout.

In another aspect, there is provided the use of the compound of Formula I as described herein or the pharmaceutical composition as described herein, for the manufacture of a medicament for the treatment of a disease or disorder selected from the group consisting of: obesity (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout.

In another aspect, there is provided a method for the treatment of a disease or disorder selected from the group consisting of obesity, diabetes (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout, comprising administering the compound of Formula I as defined herein or the pharmaceutical composition as defined herein, to a subject in need thereof.

In some embodiments, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

In some embodiments, the co-morbidity of diabetes (e.g. type 1) is selected from diabetic nephropathy, chronic kidney disease, diabetic retinopathy, and peripheral and autonomic neuropathy.

In some embodiments, the disease or disorder is selected from diabetes (type 1 or 2), obesity, and non-alcoholic fatty liver disease (e.g. non-alcoholic steatohepatitis).

In another aspect, there is provided a process for preparing the compound of Formula I, Form C as defined herein, comprising:
suspending a first crystalline form of Formula I in water, the first crystalline form exhibiting an X-ray powder diffraction (XRPD) pattern having characteristic peaks expressed in degrees 2θ (±0.2° 2θ) at 5.89 and 17.39;
subjecting the suspension to temperature cycling, between a first temperature and a second temperature lower than the first temperature; and
retrieving the compound of Formula I as a second crystalline form exhibiting an XRPD pattern having characteristic peaks expressed in degrees 2θ (±0.2° 2θ) at 6.46, 15.88, 19.44 and 5.86.

In some embodiments, the first temperature is between 45° C. and 65° C.

In some embodiments, the first temperature is between 50° C. and 60° C.

In some embodiments, the second temperature is between 5° C. and 35° C.

In some embodiments, the second temperature is between 5° C. and 25° C.

In some embodiments, the first temperature is of about 60° C. and the second temperature is of about 25° C.

In some embodiments, the process further comprises between the temperature cycling and the retrieval of the second crystalline form: stirring and maintaining the suspension at the second temperature.

In some embodiments, stirring and maintaining the suspension at the second temperature is performed for about 1 hour to about 12 hours.

In some embodiments, stirring and maintaining the suspension at the second temperature is performed for about 3 hours to about 6 hours.

In some embodiments, retrieving the compound of Formula I as the second crystalline form comprising filtering the suspension.

In another aspect, there is provided a process for preparing the compound of Formula I, Form C as described herein, comprising:
solubilizing a compound of Formula I in a solvent selected from the group consisting of methanol, ethanol, n-propanol and isopropanol, to obtain a solution;
adding water to the solution until a solid precipitates; and
retrieving the compound of Formula I as a crystalline form exhibiting an XRPD pattern having characteristic peaks expressed in degrees 2θ (±0.2° 2θ) at 6.46, 15.88, 19.44 and 5.86.

In some embodiments, the solvent is selected from the group consisting of methanol, ethanol and n-propanol.

In some embodiments, the solvent is ethanol.

In some embodiments, solubilizing the compound of Formula I in the solvent comprises solubilizing the compound of Formula I as a crystalline form exhibiting an X-ray powder diffraction (XRPD) pattern having characteristic peaks expressed in degrees 2θ (±0.2° 2θ) at 5.89 and 17.39.

In some embodiments, solubilizing the compound of Formula I in the solvent is performed at a first temperature between about 30° C. and a boiling point of the solvent.

In some embodiments, the first temperature is between about 35° C. and about 60° C. In some embodiments, adding water is performed at the first temperature.

In some embodiments, the process further comprises cooling the solvent/water mixture to a second temperature that is lower than the first temperature.

In some embodiments, the second temperature is between about 5° C. and about 30° C.

In some embodiments, the second temperature is between about 20° C. and about 30° C.

In some embodiments, the solvent: water ratio (by volume) is between about 1:1 and about 3:1.

In some embodiments, the solvent: water ratio is between about 2:1 and about 3:1.

In some embodiments, retrieving the compound of Formula I comprises filtering and drying the compound of Formula I.

In another aspect, there is provided a process for preparing the compound of Formula I, Form C as defined herein, comprising:
providing a slurry of a first crystalline form of Formula I in a solvent selected from the group consisting of water, n-butanol and an MIBK/n-heptane mixture, the first crystalline form exhibiting an X-ray powder diffraction (XRPD) pattern having characteristic peaks expressed in degrees 2θ (±0.2° 2θ) at 5.89 and 17.39;
stirring the slurry; and
retrieving the compound of Formula I as a crystalline form exhibiting an XRPD pattern having characteristic peaks expressed in degrees 2θ (±0.2° 2θ) at 6.46, 15.88, 19.44 and 5.86.

In some embodiments, the solvent is water.

In some embodiments, the solvent is a 1:4 (v/v) MIBK:n-heptane mixture.

In some embodiments, stirring the slurry is performed at a temperature between about 50° C. and about 70° C.

In some embodiments, the temperature is between about 55° C. and about 65° C.

In some embodiments, the process further comprises adding a seed of the compound of Formula I, Form C, to the mixture.

### BRIEF DESCRIPTION OF THE FIGURES

FIG. 1A is a phase map of the anhydrate crystalline forms of the compound of Formula I;

FIG. 1B is a phase interconversion map of solvate and hydrate crystalline forms of the compound of Formula I;

FIG. 2A is an XRPD of the compound of Formula I, Form A;

FIG. 2B is a graph including TGA and DSC thermograms of Form A;

FIG. 2C is an XRPD overlay of Form A and Form J;

FIG. 3A is an XRPD overlay of Form A with Form I and Form B from temperature cycling in IPA;

FIG. 3B is a graph including TGA and DSC thermograms of Form B;

FIG. 4A is an XRPD overlay of Form A and Form C obtained from temperature cycling in H₂O;

FIG. 4B is a graph including TGA and DSC thermograms of Form C;

FIG. 5A is an XRPD overlay of Form A with Form D and Form H obtained from temperature cycling in toluene;

FIG. 5B is a graph including TGA and DSC thermograms of Form D;

FIG. 6A is an XRPD overlay of Form A with Form E and Form P obtained from temperature cycling in IPAc/MTBE (1:3 v/v);

FIG. 6B is a graph including TGA and DSC thermograms of Form E;

FIG. 6C is a graph including a cycle DSC thermogram of Form E;

FIG. 7A is an XRPD overlay of Form A with Form G and Form F from RT slurry in THF/H₂O (0.92/0.08 v/v Aw=0.2 and 0.83/0.17 v/v Aw=0.4, respectively);

FIG. 7B is a graph including TGA and DSC thermograms of Form F;

FIG. 8A is an XRPD overlay of Form A and Form K from crash cooling in anisole;

FIG. 8B is a graph including TGA and DSC thermograms of Form K;

FIG. 8C is an XRPD overlay of Form K and Form K heated to 90° C. and cooled back to RT;

FIG. 9A is an XRPD overlay of Form A and Form L from crash cooling in anisole;

FIG. 9B is a graph including TGA and DSC thermograms of Form L;

FIG. 10A is an XRPD overlay of Form A and Form M from crash cooling in IPAC;

FIG. 10B is a graph including TGA and DSC thermograms of Form M;

FIG. 11A is an XRPD overlay of Form A and Form N obtained from temperature cycling in CHCl3/n-Heptane (1:3 v/v);

FIG. 11B is a graph including TGA and DSC thermograms of Form N;

FIG. 12A is an XRPD overlay of Form A and Form O from slow evaporation in MeOH;

FIG. 12B is a graph including TGA and DSC thermograms of Form O;

FIG. 13A is an XRPD overlay of Form A and Form Q from slow evaporation in slurrying Form A in n-Butanol at 100° C.;

FIG. 13B is a graph including TGA and DSC thermograms of Form Q;

FIG. 14 is an XRPD overlay of the 5-gram scale up batch showing that Form C is obtained; and

FIG. 15 is an overlay of a simulated XRPD from single crystal X-ray diffraction data and the experimental XRPD pattern of Form B.

### DETAILED DESCRIPTION

### Definitions

The term “stable”, as used herein, includes chemical stability and/or solid-state stability. A compound is considered chemically stable when the compound can be stored in an isolated solid form, or in the form of a solid formulation in which it may be provided in admixture with pharmaceutically acceptable carriers, diluents or adjuvants, under normal storage conditions, without any significant degree of chemical degradation or decomposition.

A compound is considered to have solid-state stability when the compound can be stored in an isolated solid form, or in the form of a solid formulation in which it may be provided in admixture with pharmaceutically acceptable carriers, diluents or adjuvants, under normal storage conditions, without any significant degree of solid state transformation (e.g. crystallisation, recrystallisation, loss of crystallinity, solid state phase transition, hydration, dehydration, deliquescence, solvation or desolvation).

Crystalline forms of solid chemical compounds influence not only their dissolution behavior (i.e. bioavailability) but also their solid-state stability. One way of comparing the solid-state stability of crystalline forms is to evaluate the relative “thermodynamic stability” of the crystalline forms. To evaluate the thermodynamic stability of crystalline forms, typical techniques include, but are not limited to, slurrying, slow evaporation, slow cooling, slow antisolvent addition, or a combination of these methods. calorimetry techniques (e.g., Differential Scanning calorimetry) can also be used to measure thermal events and phase transitions across a wide temperature range, and a comparison between the crystalline forms can give an indication as to their relative thermodynamic stability.

The expression “pharmaceutically acceptable carrier or excipient”, as used herein, includes without limitation any adjuvant, carrier, glidant, sweetening agent, diluent, preservative, dye/colorant, flavor enhancer, surfactant, wetting agent, dispersing agent, suspending agent, stabilizer, isotonic agent, solvent or emulsifier which is known as being acceptable for pharmaceutical use in humans or domestic animals.

The expression “pharmaceutical composition”, as used herein, refers to the formulation of a compound and a pharmaceutically acceptable carrier or excipient.

The term “about”, as used herein, generally means within an acceptable standard error of the mean, when considered by a person skilled in the art. For example, depending on the value or range considered, the term “about” can mean within 10%, within 5%, or within 1% of the value or range.

As used herein, the term “hydrate” refers to a crystalline form of a molecule that further comprises molecules of water incorporated into the crystalline lattice structure. The water molecules in the hydrate may be present in a regular arrangement and/or a non-ordered arrangement. The hydrate may comprise either a stoichiometric or nonstoichiometric amount of the water molecules. For example, a hydrate with a nonstoichiometric amount of water molecules may result from partial loss of water from the hydrate.

As used herein, the terms “anhydrate” or “anhydrous” refer to a crystalline form of a molecule per se that does not further comprise molecules of water incorporated into the crystalline lattice structure.

As used herein, the term “solvate” refers to a crystalline form of a molecule that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. The solvent can include various organic solvents. It should also be understood that a “solvate” can include a single solvent, a mixture of solvents or a mixture of a solvent (or solvents) and water.

The term “substantially the same”, used herein to describe X-ray diffraction patterns, is meant to include patterns in which peaks are within a standard deviation of +0.2° 20 or an X-ray diffraction pattern comprising least 4, 5, 6, 7, 8, 9, 10, 11, 12, 13, 14, 15 or 16 peaks in common with the referenced pattern. Further, a person skilled in the art will appreciate that relative peak intensities will show inter-apparatus variability as well as variability due to degree of crystallinity, preferred orientation, prepared sample surface, and other factors. As such, the relative peak intensities should be taken as a qualitative measure.

The present description provides crystalline Forms of the compound of Formula I:

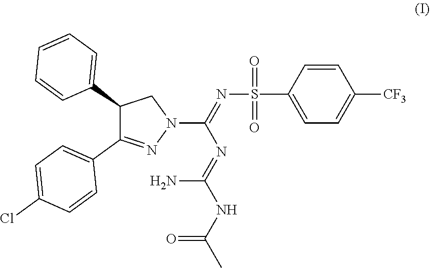 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

The structure depicted for the compound of Formula I is also meant to include all tautomeric forms of the compound of Formula I. Additionally structures depicted herein are also meant to include compounds that differ only in the presence of one or more isotopically enriched atoms. For example, compounds having the structure of the compound of Formula I except for the replacement of hydrogen by deuterium or tritium, or the replacement of a carbon by a ¹³C- or ¹⁴C-enriched carbon are within the scope of the present description.

The term “substantially pure”, when used in reference to a crystalline form of the compound of Formula I, is meant to include a crystalline form which has a purity that is greater than about 90%. This means that the crystalline form may not contain more than about 10% of any other compound, and in particular, does not contain more than about 10% of any other crystalline form of the compound of Formula I. Preferably, the term “substantially pure” means a crystalline form which has a purity that is greater than about 95%. This means that the crystalline form may not contain more than about 5% of any other compound, and in particular, does not contain more than about 5% of any other crystalline form of the compound of Formula I. More preferably, the term “substantially pure” means a crystalline form which has a purity that is greater than about 99%. This means that the crystalline form may not contain more than about 1% of any other compound, and in particular, does not contain more than about 1% of any other crystalline form of the compound of Formula I.

The term “solid” or “solid mixture” when used in reference to the compound of Formula I, refers to a mixture of crystalline forms. For example, a solid or solid mixture can include at least two different crystalline forms of the compound of Formula I. For example, a solid mixture can include crystalline Form C and one or more additional crystalline form(s) such as Form A, Form B, Form D, Form E, Form F, Form G, Form H, Form I, Form J, Form K, Form L, Form M, Form N, Form O, Form P and/or Form Q.

XRPD data were obtained using Panalytical™ X-ray powder diffractometers, used in reflection mode. The radiation used was Cu Kα (λ=1.540598 Å). It should be understood that the 2θ values listed herein are dependent on the Form of radiation used, and that a person skilled in the art would understand that the XRPD of a given crystalline form will exhibit different 2θ values if a different radiation is used (e.g., a molybdenum radiation).

The term “optically pure”, as used herein, refers to compounds which include a proportion of the desired enantiomer that is greater than that of the other enantiomer. An optically pure compound is generally made up of at least about 90%, 95% or 99% of the desired enantiomer, based upon 100 wt % total weight of the compound.

As used herein the terms “crystalline Form” or “polymorph” refers to crystal structure of a compound, having the same chemical composition but different spatial arrangements of the molecules, atoms, and/or ions forming the crystal structure.

17 crystalline Forms are obtained from polymorph screening of the compound of Formula I, including Forms A to Q. Some of the crystalline Forms can convert to other crystalline Forms, as will be described in detail herein.

### Form A

Crystalline Form A is an anhydrate. According to DSC, Form A has an endotherm that has an onset of about 152.2° C. and a peak temperature at about 162.3° C. The TGA analysis of Form A shows a weight loss of about 0.01% up to 200° C.

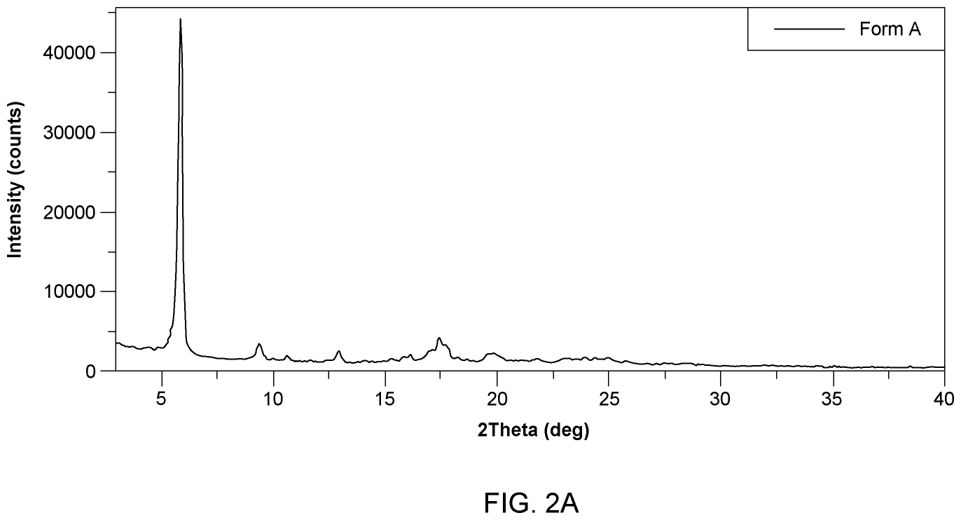

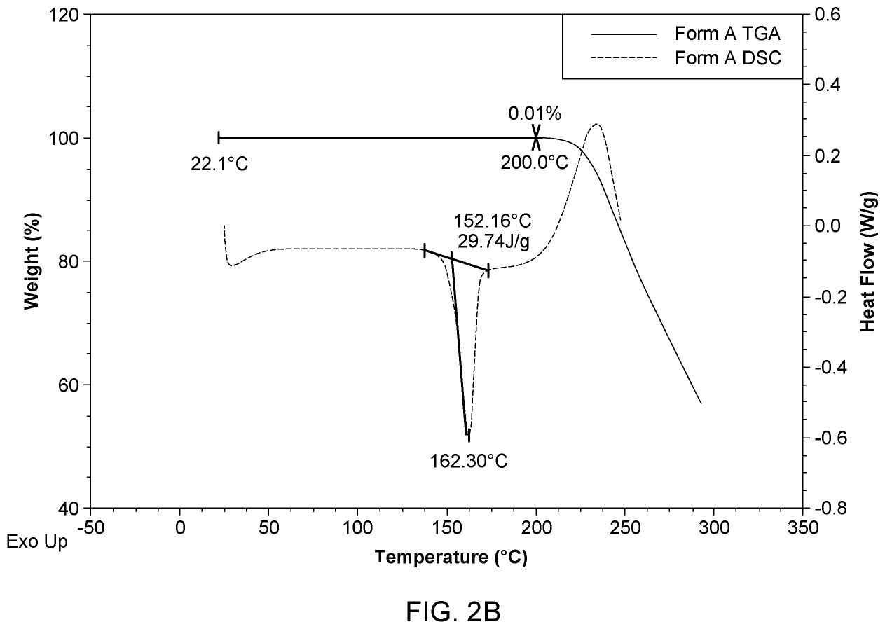

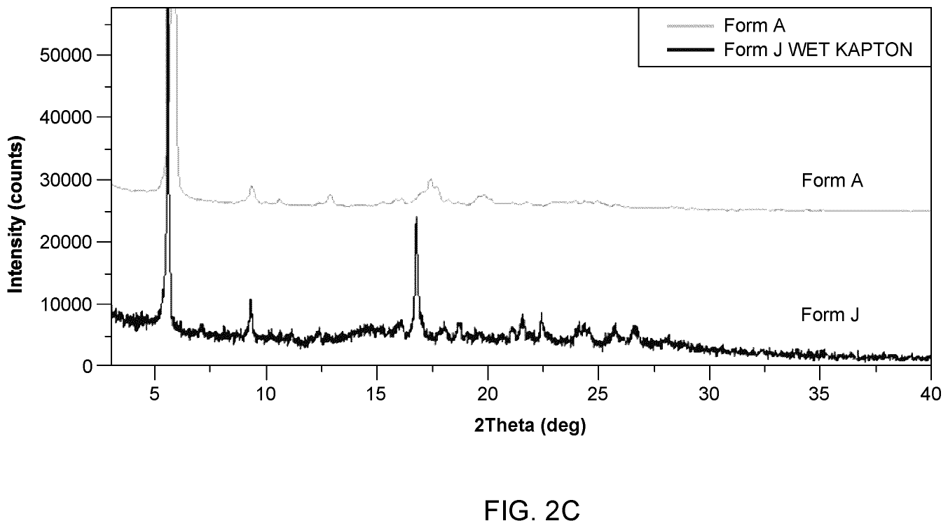

Form A has an XRPD pattern substantially the same to that shown at FIG. 2A. Form A exhibits an XRPD pattern having characteristic peaks expressed in degrees 2θ (±0.2° 2θ) at 5.89 and 17.39. The XRPD pattern of Form A can also exhibit further characteristic peaks expressed in degrees 2θ (±0.2° 2θ) at 9.39 and 17.80. The XRPD pattern of Form A can also exhibit a further characteristic peak expressed in degrees 2θ (±0.2° 2θ) at 12.96. The XRPD pattern of Form A can also exhibit a further characteristic peak expressed in degrees 2θ (±0.2° 2θ) at 19.85. The XRPD pattern of Form A can also exhibit a further characteristic peak expressed in degrees 2θ (±0.2° 2θ) at 16.96.

Form A can be prepared by anti-solvent addition of water into a solution of the compound of Formula I in acetonitrile. Form A can also be prepared by anti-solvent addition of water into a solution of the compound of Formula I in acetone. Form A can also be prepared by anti-solvent addition of heptane into a solution of the compound of Formula I in acetone. Form A can be prepared by anti-solvent addition of MTBE into a solution of the compound of Formula I in acetonitrile.

Form A can be prepared by dissolving the compound of Formula I into a solvent and evaporating the solvent until crystalline material forms (e.g., slow evaporation crystallization). The solvent can for example be selected from the group consisting of DCM, CHCl3, MEK and acetonitrile.

Form A can also be prepared by slow cooling of a solution of the compound of Formula I in a solvent or solvent mixture selected from the group consisting of IPA, toluene, MTBE, EtOH/n-heptane and CHCl3/MTBE. For example, the solvent mixtures can have the following ratios (v:v): EtOH/n-heptane (1:4) and CHCl3/MTBE (1:4).

Form A can also be prepared by triturating the compound of Formula I in DCM/n-heptane (e.g., 1/1 v/v). The trituration can for example be performed at a temperature between about 25° C. and about 35° C.

### Form B

Crystalline Form B is an IPA solvate. According to DSC, Form B has an endotherm that has an onset of about 126.4° C. and a peak temperature at about 136.3° C. The TGA analysis of Form B shows a weight loss of about 5.34% up to 174.0° C.

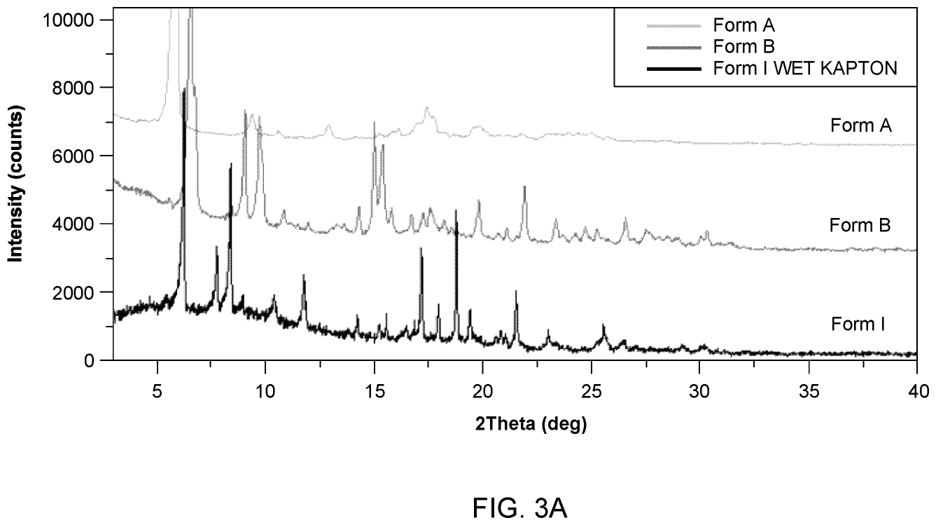

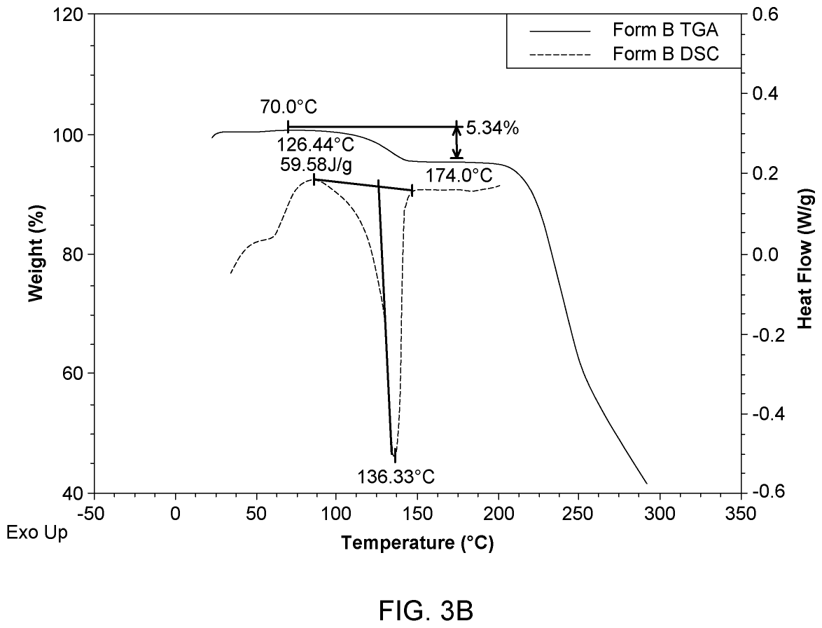

Form B has an XRPD pattern substantially the same to that shown at FIG. 3A (Form B). Form B exhibits an XRPD pattern having characteristic peaks expressed in degrees 2θ (±0.2° 2θ) at 6.60, 6.83 and 15.02. The XRPD pattern of Form B can also exhibit further characteristic peaks expressed in degrees 2θ (±0.2° 2θ) at 9.09 and 9.73. The XRPD pattern of Form B can also exhibit further characteristic peaks expressed in degrees 2θ (±0.2° 2θ) at 15.40 and 9.88. The XRPD pattern of Form B can also exhibit further characteristic peaks expressed in degrees 2θ (±0.2° 2θ) at 21.90, 19.84 and 14.32. The XRPD pattern of Form B can also exhibit further characteristic peaks expressed in degrees 2θ (±0.2° 2θ) at 26.56, 15.82 and 23.39.

### Form C

Crystalline Form C is an anhydrate. According to DSC, Form C has an endotherm that has an onset of about 157.1° C. and a peak temperature at about 167.9° C. The TGA analysis of Form C shows a weight loss of about 0.32% up to 175.0° C.

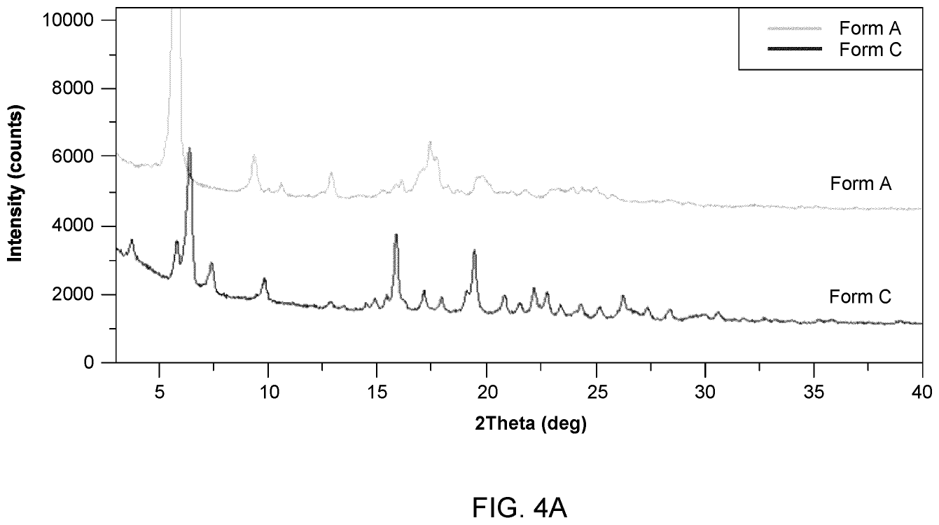

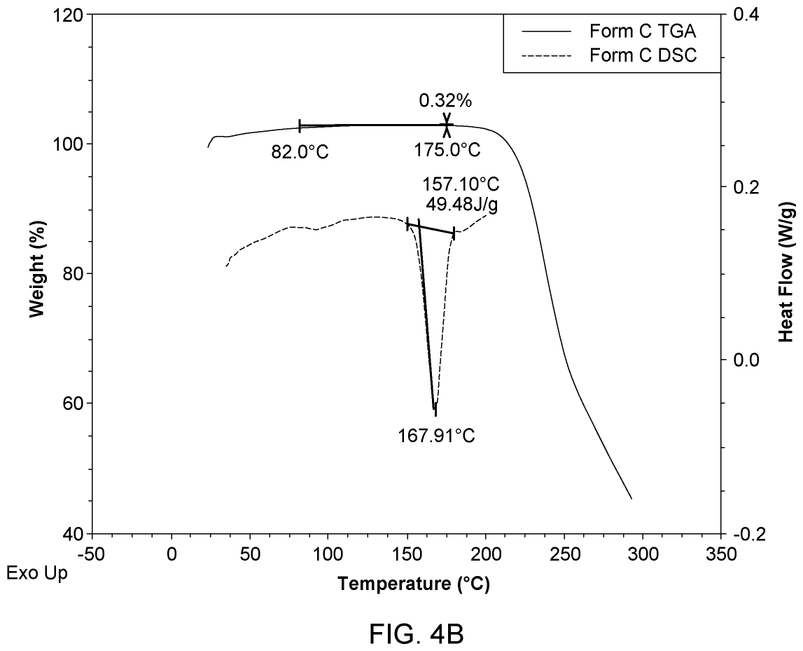

Form C has an XRPD pattern substantially the same to that shown at FIG. 4A (Form C). Form C exhibits an XRPD pattern having characteristic peaks expressed in degrees 2θ (±0.2° 2θ) at 6.46, 15.88, 19.44 and 5.86. The XRPD pattern of Form C can also exhibit further characteristic peaks expressed in degrees 2θ (±0.2° 2θ) at 7.46, 22.15 and 26.24. The XRPD pattern of Form C can also exhibit further characteristic peaks expressed in degrees 2θ (±0.2° 2θ) at 3.81, 17.17 and 20.84. The XRPD pattern of Form C can also exhibit further characteristic peaks expressed in degrees 2θ (±0.2° 2θ) at 17.95, 15.43 and 24.24. The XRPD pattern of Form C can also exhibit a further characteristic peak expressed in degrees 2θ (±0.2° 2θ) at 21.53, 25.11 and 23.34. The XRPD pattern of Form C can also exhibit a further characteristic peak expressed in degrees 2θ (±0.2° 2θ) at 14.94, 27.38 and 28.41. The XRPD pattern of Form C can also exhibit a further characteristic peak expressed in degrees 2θ (±0.2° 2θ) at 30.57, 12.94 and 14.50.

Form C can be prepared by suspending the compound of Formula I, Form A in water; subjecting the suspension to temperature cycling, between a first temperature and a second temperature lower than the first temperature; and retrieving Form C. In some embodiments, the first temperature is between 45° C. and 65° C., or between about 50° C. and about 60° C. In some embodiments, the second temperature is between about 5° C. and about 35° C., or between about 5° C. and about 25° C. In some embodiments, the first temperature is of about 60° C. and the second temperature is of about 25° C. In some embodiments, preparing Form C further includes stirring and maintaining the suspension at the second temperature between the temperature cycling and the retrieval of Form C. In some embodiments, stirring and maintaining the suspension at the second temperature is performed for about 1 hour to about 12 hours, or for about 3 hours to about 6 hours, or for about 4.5 hours. In some embodiments, preparing Form C further includes retrieving the compound of Formula I as the second crystalline form comprising filtering the suspension.

Form C can also be prepared by solubilizing a compound of Formula I in a solvent selected from the group consisting of methanol, ethanol, n-propanol and isopropanol, to obtain a solution; adding water to the solution until a solid precipitates; and retrieving the precipitated solid as Form C. In some embodiments, the solvent is selected from the group consisting of methanol, ethanol and n-propanol. In some embodiments, the solvent is ethanol. In some embodiments, wherein solubilizing the compound of Formula I in the solvent comprises solubilizing the compound of Formula I as Form A. In some embodiments, solubilizing the compound of Formula I in the solvent is performed at a first temperature between about 30° C. and a boiling point of the solvent. In some embodiments, the first temperature is between about 35° C. and about 60° C. In some embodiments, adding water is performed at the first temperature. In some embodiments, preparing Form C further includes cooling the solvent/water mixture to a second temperature that is lower than the first temperature. In some embodiments, the second temperature is between about 5° C. and about 30° C., or between about 20° C. and about 30° C. In some embodiments, the solvent: water ratio (v/v) is between about 1:1 and about 3:1, or between about 2:1 and about 3:1. In some embodiments, retrieving the compound of Formula I, Form C includes filtering and drying.

Form C can also be prepared by providing a slurry of the Compound of Formula I, Form A in a solvent selected from the group consisting of water and an MIBK/n-heptane mixture; stirring the slurry; and retrieving the compound of Formula I as Form C. In some embodiments, the solvent in water. In other embodiments, the solvent a a 1:4 (v/v) MIBK:n-heptane mixture. In some embodiments, stirring the slurry is performed at a temperature between about 50° C. and about 70° C., or between about 55° C. and about 65° C.

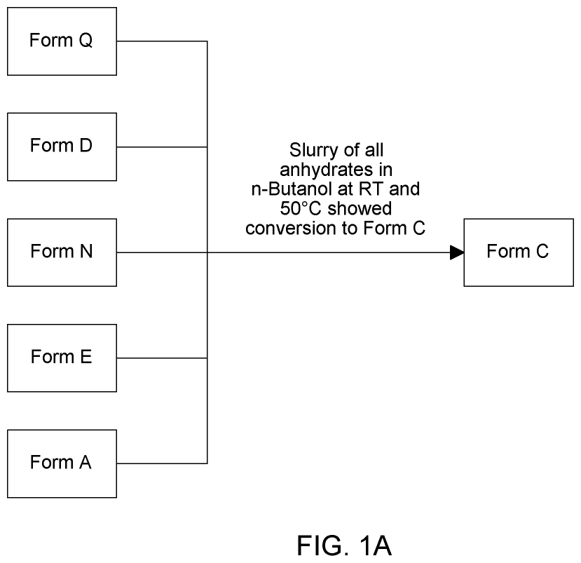

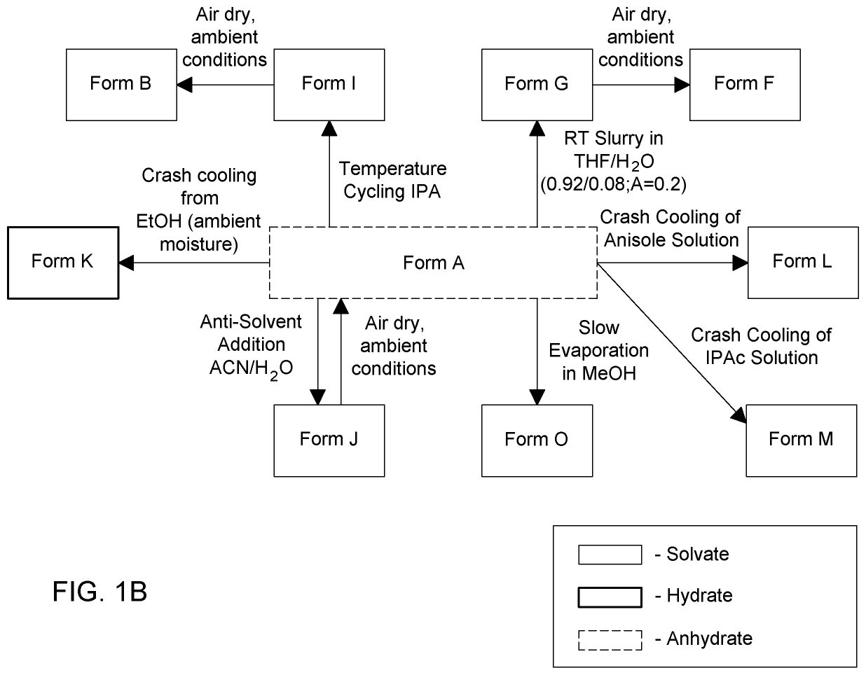

As shown at FIG. 1A, Form C can also be prepared by slurrying any one of the anhydrate crystalline Forms A, D, E, N and Q in n-butanol at 25° C. or at 50° C. converts the respective anhydrate crystalline forms to Form C.

In some embodiments, a seed of Form C can be added to any one of the manufacturing process of Form C described herein.

### Form D

Crystalline Form D is an anhydrate. According to DSC, Form D has an endotherm that has an onset of about 155.9° C. and a peak temperature at about 163.9° C. The TGA analysis of Form D shows a weight loss of about 0.07% up to 180.0° C.

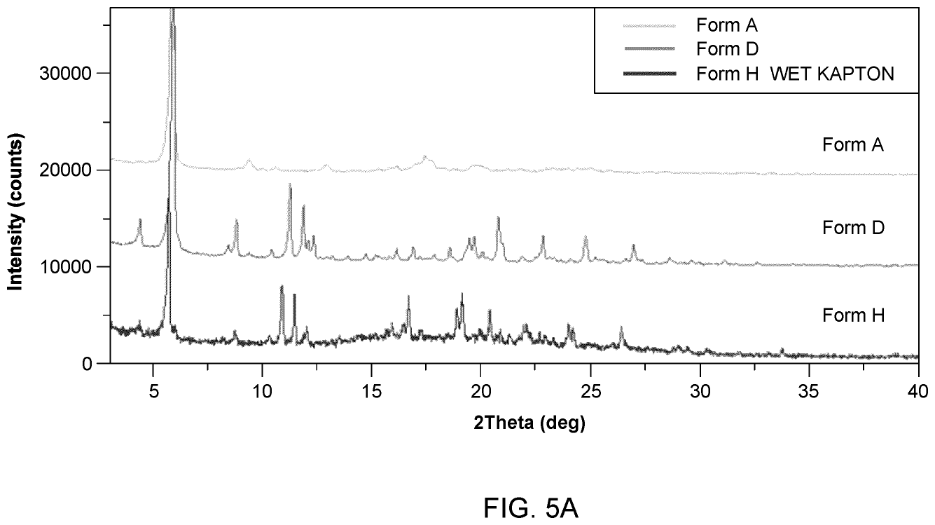

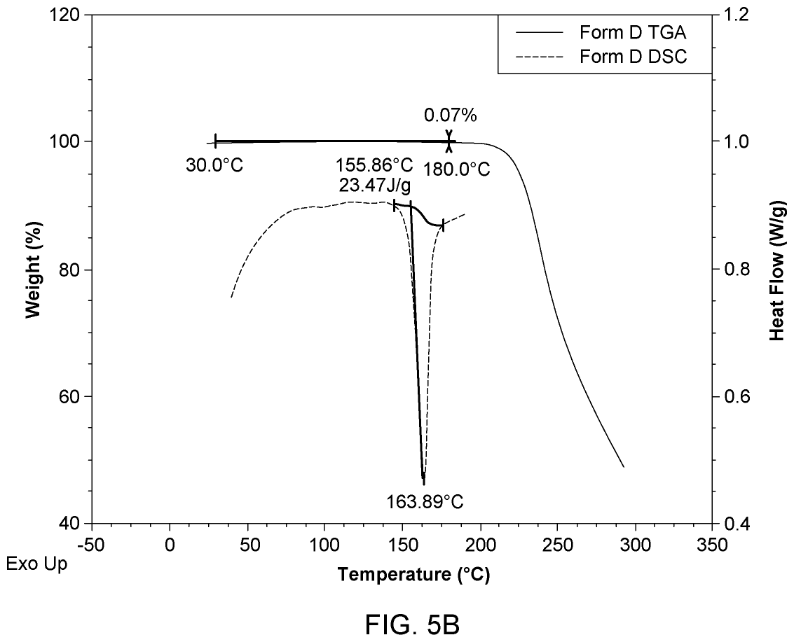

Form D has an XRPD pattern substantially the same to that shown at FIG. 5A (Form D). Form D exhibits an XRPD pattern having characteristic peaks expressed in degrees 2θ (±0.2° 2θ) at 5.94, 11.27 and 11.86. The XRPD pattern of Form D can also exhibit further characteristic peaks expressed in degrees 2θ (±0.2° 2θ) at 20.75, 8.82 and 24.74. The XRPD pattern of Form D can also exhibit further characteristic peaks expressed in degrees 2θ (±0.2° 2θ) at 4.43, 22.79 and 19.66. The XRPD pattern of Form D can also exhibit further characteristic peaks expressed in degrees 2θ (±0.2° 2θ) at 19.41, 12.34 and 26.91. The XRPD pattern of Form D can also exhibit a further characteristic peak expressed in degrees 2θ (±0.2° 2θ) at 12.08 and 20.98. The XRPD pattern of Form D can also exhibit a further characteristic peak expressed in degrees 2θ (±0.2° 2θ) at 18.53 and 16.89.

### Form E

Crystalline Form E is an anhydrate. According to DSC, Form E has two endotherms, with onsets of 120.6° C. and 146.0° C. and peak temperatures of 129.5° C. and 154.6° C. respectively. The TGA analysis of Form E shows a weight loss of about 0.317% up to 177.9° C.

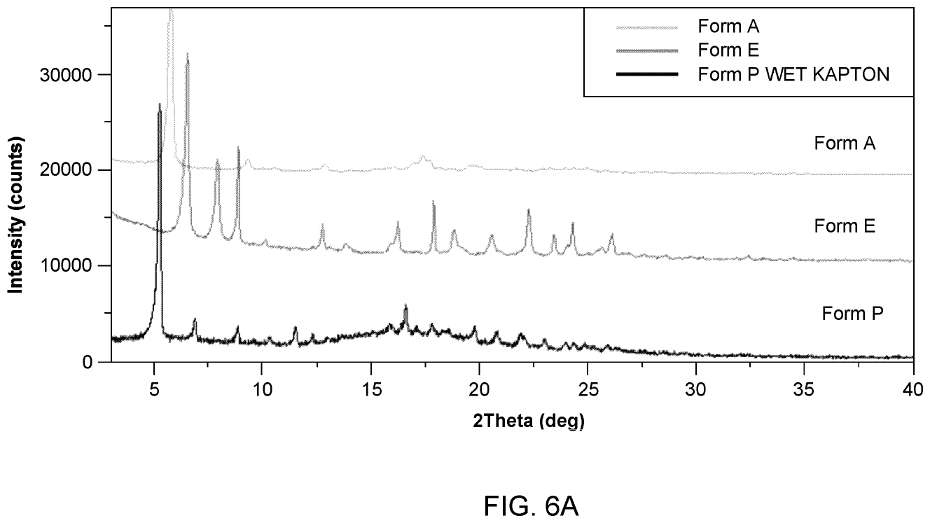

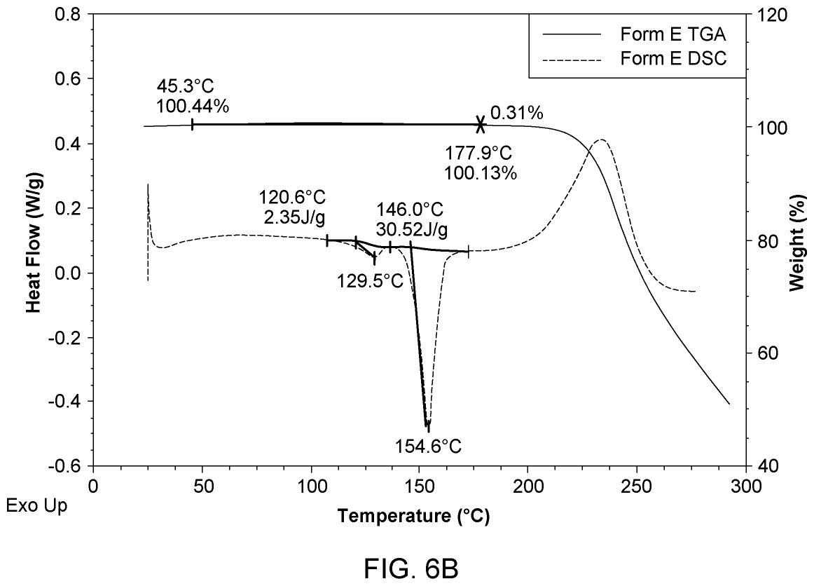

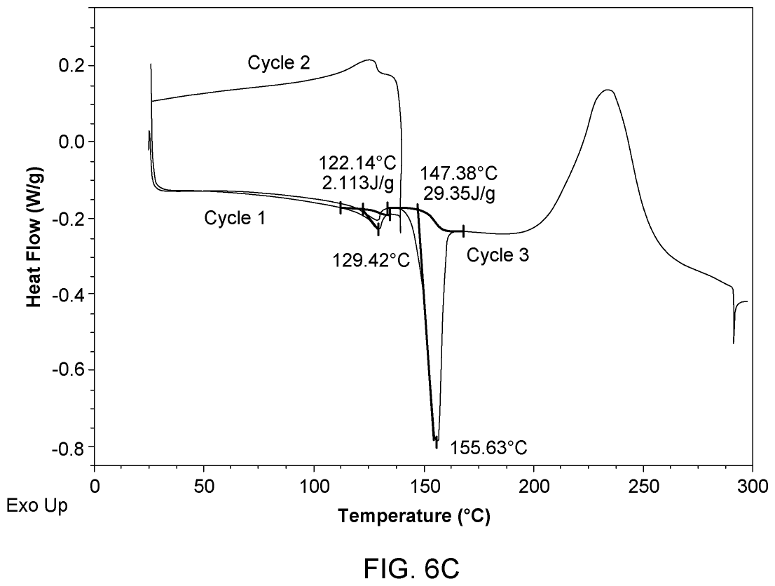

Form E has an XRPD pattern substantially the same to that shown at FIG. 6A (Form E). Form E exhibits an XRPD pattern having characteristic peaks expressed in degrees 2θ (±0.2° 2θ) at 6.62, 8.95 and 8.01. The XRPD pattern of Form E can also exhibit further characteristic peaks expressed in degrees 2θ (±0.2° 2θ) at 17.94, 22.30 and 24.31. The XRPD pattern of Form E can also exhibit further characteristic peaks expressed in degrees 2θ (±0.2° 2θ) at 16.29, 12.82 and 18.83. The XRPD pattern of Form E can also exhibit further characteristic peaks expressed in degrees 2θ (±0.2° 2θ) at 23.45, 26.11 and 20.62. The XRPD pattern of Form E can also exhibit a further characteristic peak expressed in degrees 2θ (±0.2° 2θ) at 15.95 and 13.86. The XRPD pattern of Form E can also exhibit a further characteristic peak expressed in degrees 2θ (±0.2° 2θ) at 25.65 and 10.22.

### Form F

Crystalline Form F is a THF solvate. According to DSC, Form F has a broad endotherm that has an onset of about 119.5° C. and a peak temperature at about 132.4° C. The TGA analysis of Form F shows a weight loss of about 1.85% up to 165.0° C.

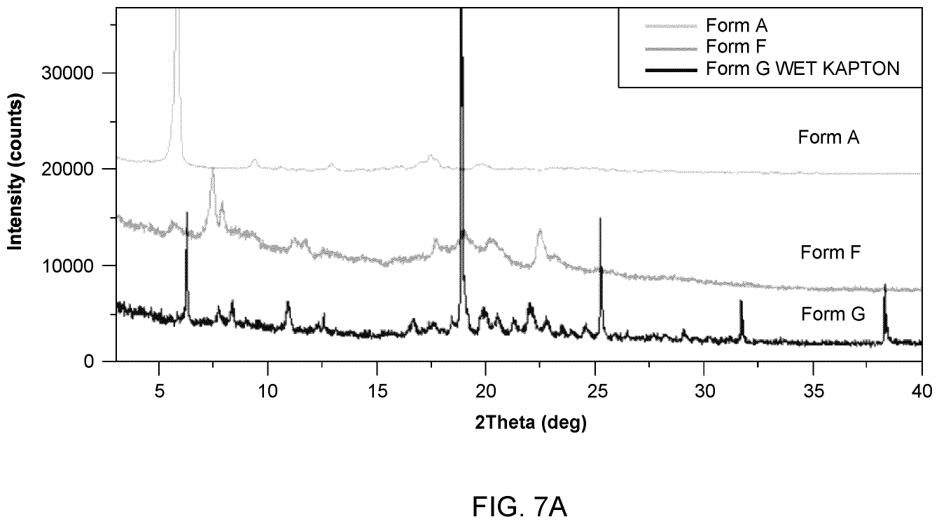

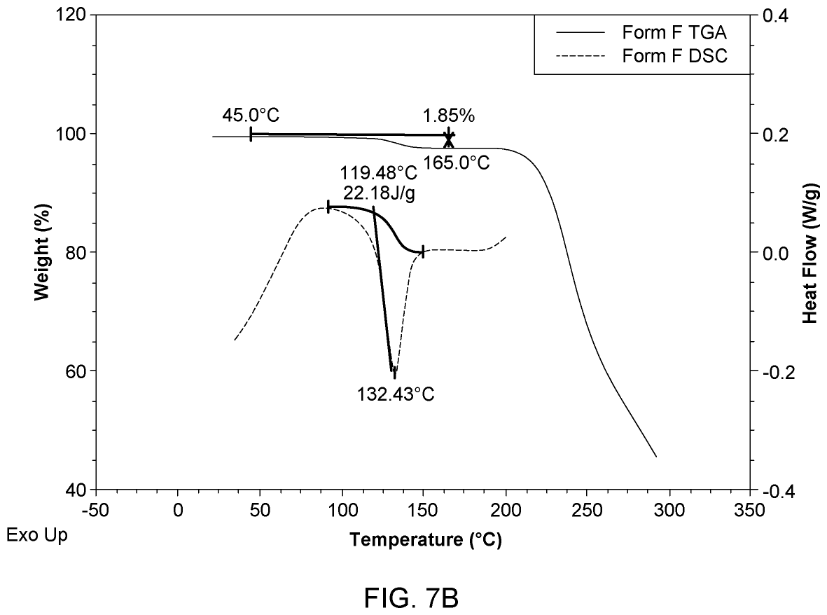

Form F has an XRPD pattern substantially the same to that shown at FIG. 7A (Form F). Form F exhibits an XRPD pattern having characteristic peaks expressed in degrees 2θ (±0.2° 2θ) at 7.51, 22.37 and 7.89. The XRPD pattern of Form F can also exhibit further characteristic peaks expressed in degrees 2θ (±0.2° 2θ) at 18.95, 20.25, 17.62 and 11.22. The XRPD pattern of Form F can also exhibit further characteristic peaks expressed in degrees 2θ (±0.2° 2θ) at 11.73, 23.13, 9.26 and 5.66. The XRPD pattern of Form F can also exhibit further characteristic peaks expressed in degrees 2θ (±0.2° 2θ) at 12.53, 9.64, 15.69 and 4.17. The XRPD pattern of Form F can also exhibit a further characteristic peak expressed in degrees 2θ (±0.2° 2θ) at 19.91, 14.32, 12.22 and 13.51.

### Form G

Crystalline Form G is a labile solvate that converts to Form F upon drying.

Form G has an XRPD pattern substantially the same to that shown at FIG. 7A (Form G). Form G exhibits an XRPD pattern having characteristic peaks expressed in degrees 2θ (±0.2° 2θ) at 18.86, 25.22 and 6.28. The XRPD pattern of Form G can also exhibit further characteristic peaks expressed in degrees 2θ (±0.2° 2θ) at 38.23, 31.67 and 21.95. The XRPD pattern of Form G can also exhibit further characteristic peaks expressed in degrees 2θ (±0.2° 2θ) at 10.88 and 8.36. The XRPD pattern of Form G can also exhibit further characteristic peaks expressed in degrees 2θ (±0.2° 2θ) at 19.75 and 12.56. The XRPD pattern of Form G can also exhibit a further characteristic peak expressed in degrees 2θ (±0.2° 2θ) at 20.51 and 22.74.

### Form H

Crystalline Form H is a labile solvate that converts to Form D upon drying.

Form H has an XRPD pattern substantially the same to that shown at FIG. 5A (Form H). Form H exhibits an XRPD pattern having characteristic peaks expressed in degrees 2θ (±0.2° 2θ) at 5.73, 10.90, and 11.45. The XRPD pattern of Form H can also exhibit further characteristic peaks expressed in degrees 2θ (±0.2° 2θ) at 19.12, 16.68 and 18.86. The XRPD pattern of Form H can also exhibit further characteristic peaks expressed in degrees 2θ (±0.2° 2θ) at 20.35, 23.96, 26.36 and 22.03. The XRPD pattern of Form H can also exhibit further characteristic peaks expressed in degrees 2θ (±0.2° 2θ) at 24.18, 12.02, 22.62 and 15.90. The XRPD pattern of Form H can also exhibit further characteristic peaks expressed in degrees 2θ (±0.2° 2θ) at 16.43, 6.01, 8.73, 20.84 and 4.36.

### Form I

Crystalline Form I is a labile solvate that converts to Form B upon drying.

Form I has an XRPD pattern substantially the same to that shown at FIG. 3A (Form I). Form I exhibits an XRPD pattern having characteristic peaks expressed in degrees 2θ (±0.2° 2θ) at 6.28, 8.43 and 18.77. The XRPD pattern of Form I can also exhibit further characteristic peaks expressed in degrees 2θ (±0.2° 2θ) at 17.18, 7.78 and 21.51. The XRPD pattern of Form I can also exhibit further characteristic peaks expressed in degrees 2θ (±0.2° 2θ) at 11.77, 19.40 and 17.96. The XRPD pattern of Form I can also exhibit further characteristic peaks expressed in degrees 2θ (±0.2° 2θ) at 10.39, 25.55 and 15.56. The XRPD pattern of Form I can also exhibit further characteristic peaks expressed in degrees 2θ (±0.2° 2θ) at 9.00, 14.23, 23.00 and 15.25. The XRPD pattern of Form I can also exhibit further characteristic peaks expressed in degrees 2θ (±0.2° 2θ) at 20.84, 16.47, 21.02 and 26.47.

### Form J

Crystalline Form J is a labile solvate that converts to Form J upon drying.

Form J has an XRPD pattern substantially the same to that shown at FIG. 2C (Form J). Form J exhibits an XRPD pattern having characteristic peaks expressed in degrees 2θ (±0.2° 2θ) at 5.59 and 16.76. The XRPD pattern of Form J can also exhibit further characteristic peaks expressed in degrees 2θ (±0.2° 2θ) at 9.35 and 22.43. The XRPD pattern of Form J can also exhibit further characteristic peaks expressed in degrees 2θ (±0.2° 2θ) at 22.75, 21.63 and 26.63. The XRPD pattern of Form J can also exhibit further characteristic peaks expressed in degrees 2θ (±0.2° 2θ) at 18.78, 21.11 and 24.05. The XRPD pattern of Form J can also exhibit further characteristic peaks expressed in degrees 2θ (±0.2° 2θ) at 18.10, 24.62 and 16.17.

### Form K

Crystalline Form K is a hemi-hydrate. According to DSC, Form K shows two endothermic events with onsets of 53.6° C. and 106.3° C. and peak temperatures at 68.7° C. and 125.9° C., respectively. The TGA analysis of Form K shows a weight loss of about 1.35% up to 160.0° C.

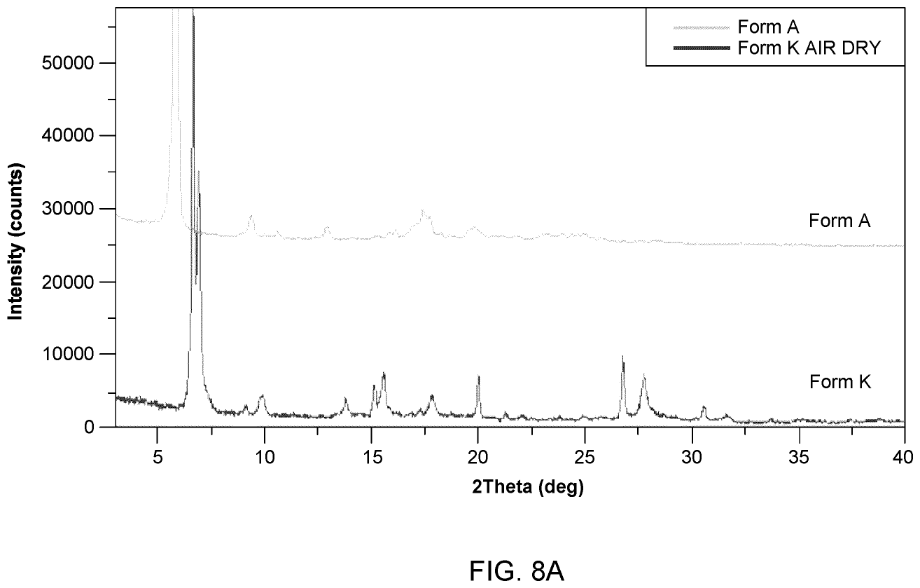

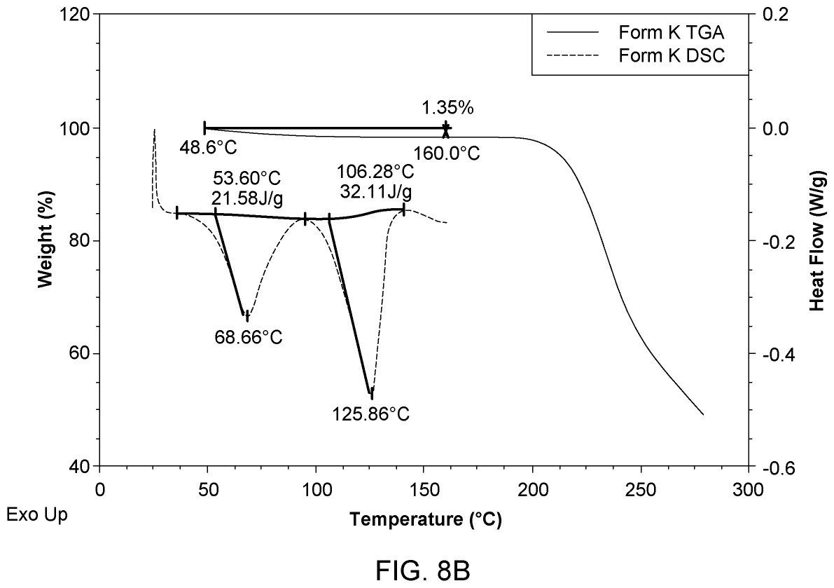

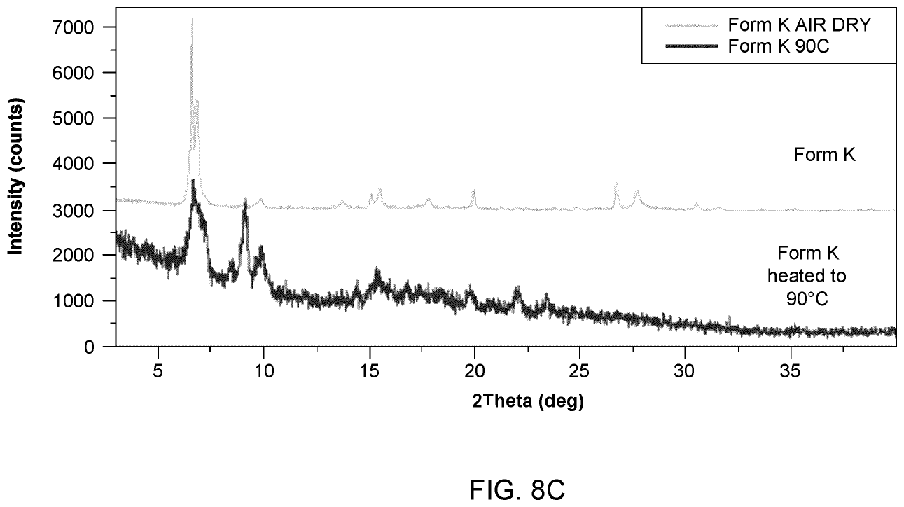

Form K has an XRPD pattern substantially the same to that shown at FIG. 8A (Form K). Form K exhibits an XRPD pattern having characteristic peaks expressed in degrees 2θ (±0.2° 2θ) at 6.65 and 6.92. The XRPD pattern of Form K can also exhibit further characteristic peaks expressed in degrees 2θ (±0.2° 2θ) at 26.74 and 20.02. The XRPD pattern of Form K can also exhibit further characteristic peaks expressed in degrees 2θ (±0.2° 2θ) at 15.55 and 27.75. The XRPD pattern of Form K can also exhibit further characteristic peaks expressed in degrees 2θ (±0.2° 2θ) at 15.13, 9.94 and 17.89. The XRPD pattern of Form K can also exhibit further characteristic peaks expressed in degrees 2θ (±0.2° 2θ) at 13.79, 30.57 and 9.13.

### Form L

Crystalline Form L is a partly desolvated anisole solvate having a stoichiometry API:Anisole of about 0:0.26. According to DSC, Form L shows a single endothermic event with an onset of 126.4° C. and a peak temperature of 133.8° C. in the DSC curve that occurs after the solvent is driven. The TGA analysis of Form L shows a weight loss of about 4.56% up to 165° C.

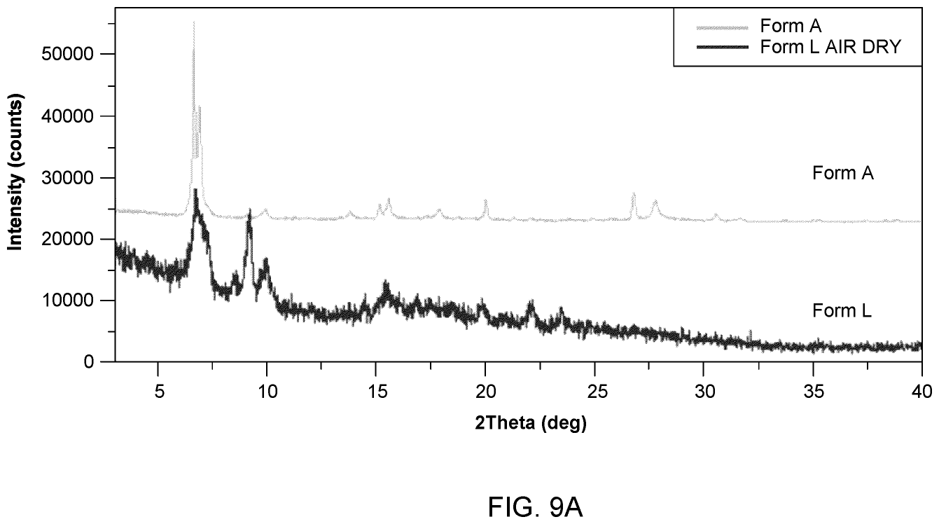

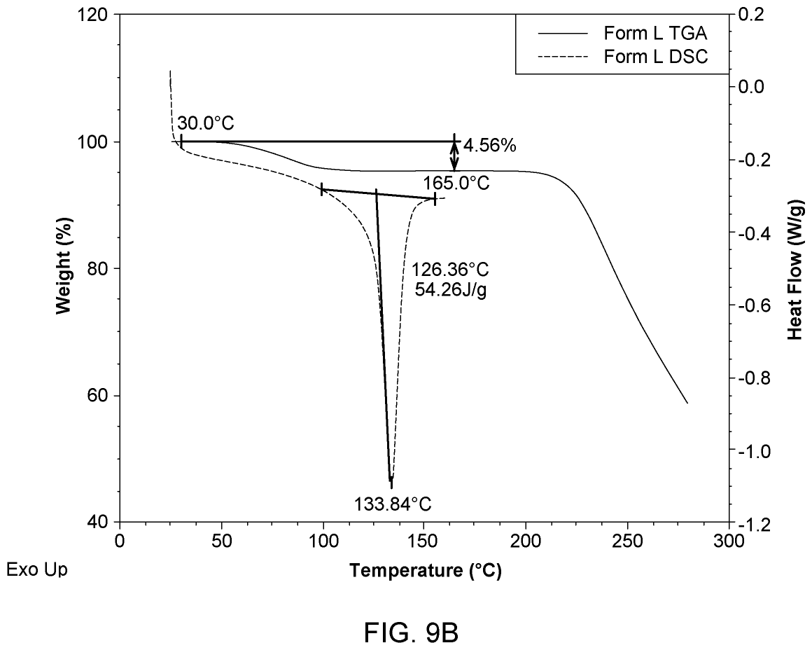

Form L has an XRPD pattern substantially the same to that shown at FIG. 9A (Form L). Form L exhibits an XRPD pattern having characteristic peaks expressed in degrees 2θ (±0.2° 2θ) at 6.07 and 6.65. The XRPD pattern of Form L can also exhibit a further characteristic peak expressed in degrees 2θ (±0.2° 2θ) at 5.24. The XRPD pattern of Form L can also exhibit a further characteristic peak expressed in degrees 2θ (±0.2° 2θ) at 20.64. The XRPD pattern of Form L can also exhibit a further characteristic peak expressed in degrees 2θ (±0.2° 2θ) at 8.03. The XRPD pattern of Form L can also exhibit a further characteristic peak expressed in degrees 2θ (±0.2° 2θ) at 22.38. The XRPD pattern of Form L can also exhibit a further characteristic peak expressed in degrees 2θ (±0.2° 2θ) at 17.99.

### Form M

Crystalline Form M is an IPAc mono-solvate. According to DSC, Form M shows 2 overlapping endothermic events with an onset of 109° C., and peak temperatures at 116.7° C. and 128.2° C. that occur concurrently with the TGA weight loss step. The TGA analysis of Form M shows a weight loss of 13.33% up to 165.0° C.

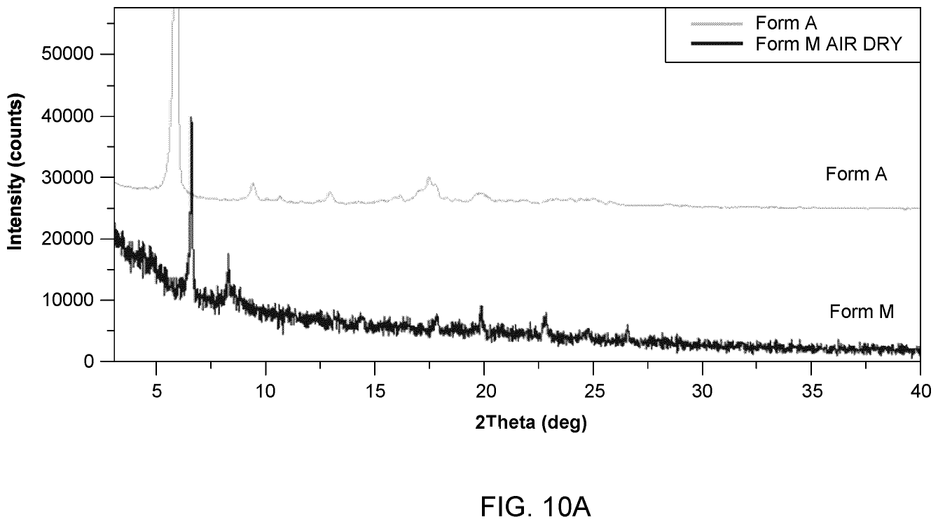

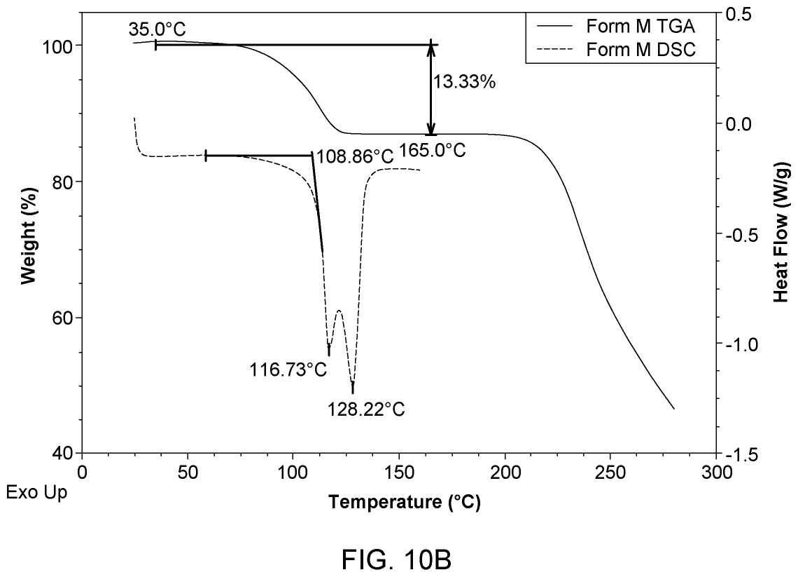

Form M has an XRPD pattern substantially the same to that shown at FIG. 10A (Form M). Form M exhibits an XRPD pattern having characteristic peaks expressed in degrees 2θ (±0.2° 2θ) at 6.59, 8.26 and 19.79. The XRPD pattern of Form M can also exhibit further characteristic peaks expressed in degrees 2θ (±0.2° 2θ) at 22.71 and 26.49. The XRPD pattern of Form M can also exhibit further characteristic peaks expressed in degrees 2θ (±0.2° 2θ) at 17.84 and 14.38. The XRPD pattern of Form M can also exhibit further characteristic peaks expressed in degrees 2θ (±0.2° 2θ) at 4.73, 24.57 and 12.25. The XRPD pattern of Form M can also exhibit further characteristic peaks expressed in degrees 2θ (±0.2° 2θ) at 20.69, 13.29 and 16.40.

### Form N

Crystalline Form N is an anhydrate. According to DSC, Form N shows a single endotherm with an onset of 141.9° C. and a peak temperature of 152.1° C. The TGA analysis of Form N shows a weight loss of 0.46% up to 179.0° C.

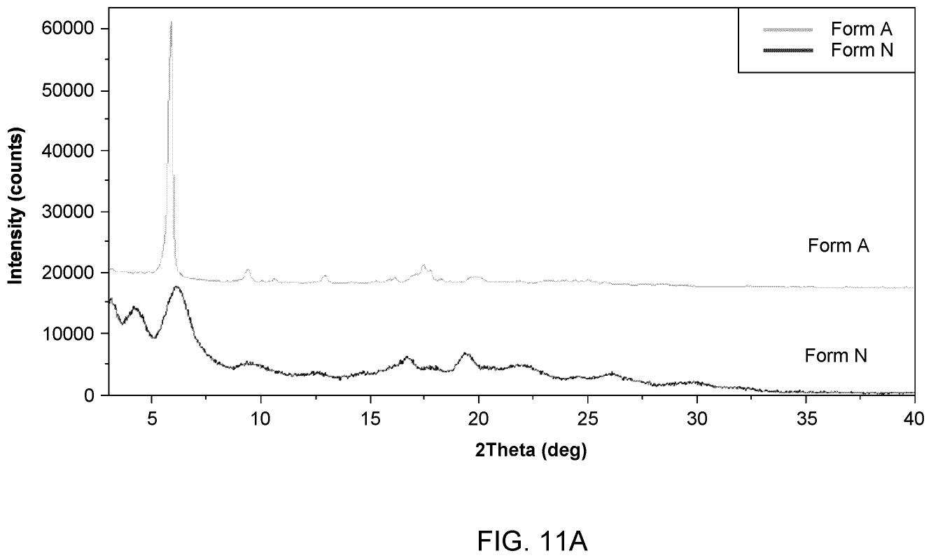

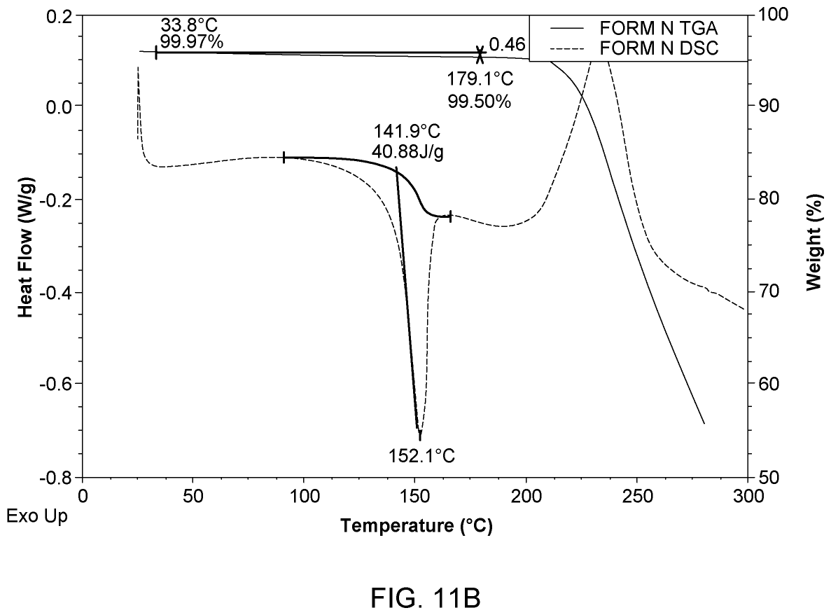

Form N has an XRPD pattern substantially the same to that shown at FIG. 11A (Form N). Form N exhibits an XRPD pattern having characteristic peaks expressed in degrees 2θ (±0.2° 2θ) at 6.12, 4.16 and 19.29. The XRPD pattern of Form N can also exhibit further characteristic peaks expressed in degrees 2θ (±0.2° 2θ) at 3.32 and 16.65. The XRPD pattern of Form N can also exhibit further characteristic peaks expressed in degrees 2θ (±0.2° 2θ) a 4.63 and 3.11. The XRPD pattern of Form N can also exhibit further characteristic peaks expressed in degrees 2θ (±0.2° 2θ) at 21.81, 22.45 and 26.20. The XRPD pattern of Form N can also exhibit further characteristic peaks expressed in degrees 2θ (±0.2° 2θ) at 9.93, 9.12 and 18.05.

### Form O

Crystalline Form O is a methanol mono-solvate. According to DSC, Form O shows a broad endotherm with an onset of 107.4° C. and a peak temperature of 110.6° C. The TGA analysis of Form O shows a weight loss of 5.11% up to 160.0° C.

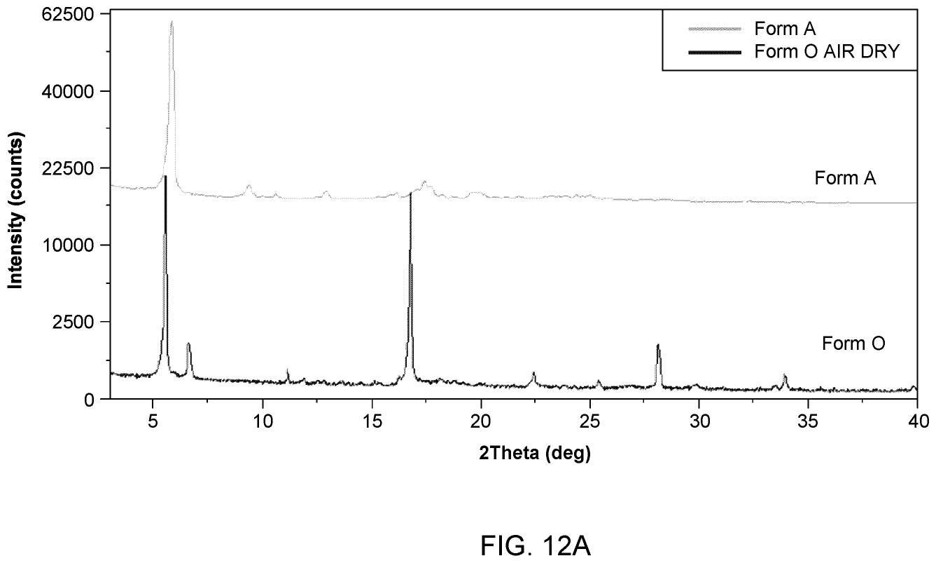

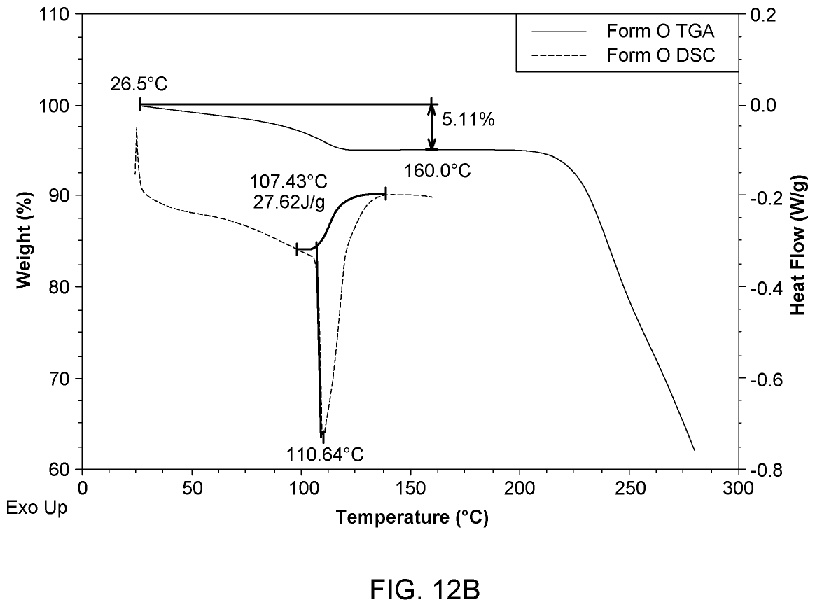

Form O has an XRPD pattern substantially the same to that shown at FIG. 12A (Form O). Form O exhibits an XRPD pattern having characteristic peaks expressed in degrees 2θ (±0.2° 2θ) at 5.59 and 16.76. The XRPD pattern of Form O can also exhibit further characteristic peaks expressed in degrees 2θ (±0.2° 2θ) at 28.10 and 6.63. The XRPD pattern of Form O can also exhibit further characteristic peaks expressed in degrees 2θ (±0.2° 2θ) at 11.16 and 22.41. The XRPD pattern of Form O can also exhibit further characteristic peaks expressed in degrees 2θ (±0.2° 2θ) at 33.87 and 25.38. The XRPD pattern of Form O can also exhibit further characteristic peaks expressed in degrees 2θ (±0.2° 2θ) at 11.89 and 16.20.

### Form P

Crystalline Form P is a labile solvate that converts to Form E upon drying.

Form P has an XRPD pattern substantially the same to that shown at FIG. 6A (Form P). Form P exhibits an XRPD pattern having characteristic peaks expressed in degrees 2θ (±0.2° 2θ) at 5.38, 16.69 and 6.93. The XRPD pattern of Form P can also exhibit further characteristic peaks expressed in degrees 2θ (±0.2° 2θ) at 11.61, 21.89 and 8.95. The XRPD pattern of Form P can also exhibit further characteristic peaks expressed in degrees 2θ (±0.2° 2θ) at 19.84 and 17.85. The XRPD pattern of Form P can also exhibit further characteristic peaks expressed in degrees 2θ (±0.2° 2θ) at 20.82 and 15.86. The XRPD pattern of Form P can also exhibit further characteristic peaks expressed in degrees 2θ (±0.2° 2θ) at 12.35 and 22.99.

### Form Q

Crystalline Form Q is an anhydrate. According to DSC, Form Q shows a single broad endotherm with an onset of 101.4° C. and a peak temperature of 130.1° C. The TGA analysis of Form N shows a weight loss 0.63% up to 160.0° C.

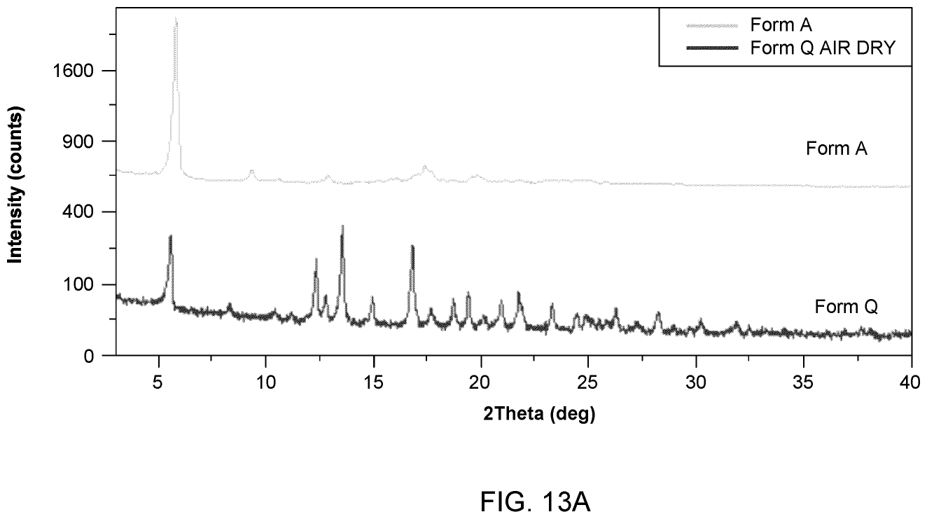

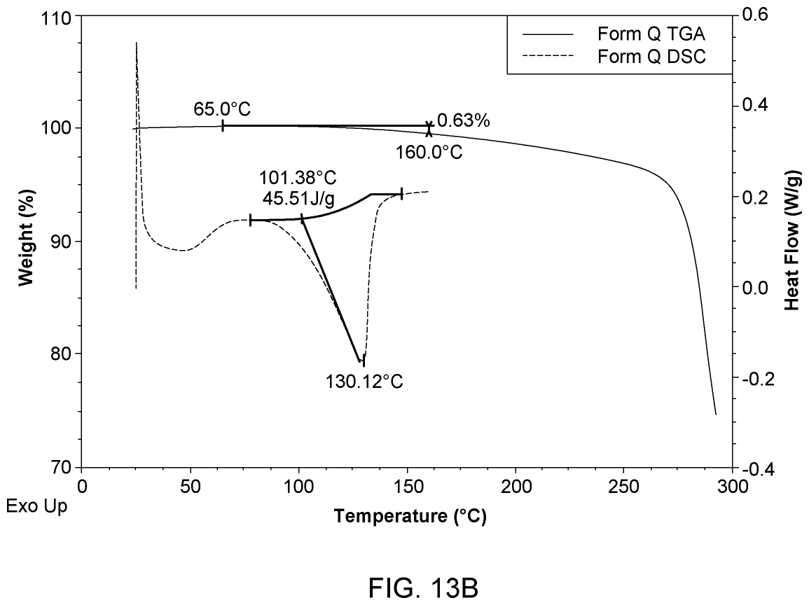

Form Q has an XRPD pattern substantially the same to that shown at FIG. 13A (Form N). Form Q exhibits an XRPD pattern having characteristic peaks expressed in degrees 2θ (±0.2° 2θ) at 13.58, 5.61 and 16.82. The XRPD pattern of Form Q can also exhibit further characteristic peaks expressed in degrees 2θ (±0.2° 2θ) at 12.34, 21.73 and 19.39. The XRPD pattern of Form Q can also exhibit further characteristic peaks expressed in degrees 2θ (±0.2° 2θ) at 14.95, 20.94 and 18.73. The XRPD pattern of Form Q can also exhibit further characteristic peaks expressed in degrees 2θ (±0.2° 2θ) at 12.78, 23.31, 26.25 and 28.21. The XRPD pattern of Form Q can also exhibit further characteristic peaks expressed in degrees 2θ (±0.2° 2θ) at 17.69, 8.37, 24.47 and 31.84.

### Formulations, Methods and Uses

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient or subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

The compound of Formula I is useful for the treatment of diseases and disorders where inhibition of the cannabinoid receptor CB₁ is indicated, for instance such as those described in U.S. Pat. No. 9,765,031. Such diseases and disorders are generally related to diabetes and metabolic disorders (e.g. metabolic syndrome). Preferably, the active ingredient selective targets the CB₁ receptor in peripheral tissue (e.g. adipose tissue, liver, muscle, lung, kidney, macrophages, pancreatic beta cells and gastrointestinal tract), while not interacting with CB₁ receptors in brain tissue, thereby avoiding or reducing CNS-related side effects.

The effect of the compound of Formula I may include reduced food intake, reduced body weight, reversed insulin and leptin resistance, reverse hepatic steatosis (fatty liver) and improved dyslipidemia. Examples of diseases and disorders to be treated include obesity, diabetes (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout. For instance, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, osteoarthritis, high cholesterol, and increased incidence of malignancies of the liver, ovaries, cervix, uterus, breasts, prostate, or gallbladder. In preferred examples, the disease or disorder include diabetes (type 1 or 2), obesity, and non-alcoholic fatty liver disease (e.g. non-alcoholic steatohepatitis). Examples of co-morbidities of diabetes (e.g. type 1) include diabetic nephropathy, chronic kidney disease, diabetic retinopathy, and peripheral and autonomic neuropathy.

The compound of Formula I and pharmaceutical compositions including the compound of Formula I may also be used in a method for preventing or reversing the deposition of adipose tissue in a subject, which is expected to contribute to a reduction of incidence or severity of obesity, which in turn would reduce the incidence or severity of associated co-morbidities.

The present description provides a method of treating a disorder (as described herein) in a subject, comprising administering to the subject identified as in need thereof, the compound of Formula I. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are determined again. The modulation (e.g., decrease) of symptoms and/or of a biomarker of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene 1,3-butylene glycol, glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming micro-encapsulated matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly-(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar Form may also be employed as fillers in soft and hard-filled gelatin capsules using such excipients as lactose or milk sugar as well as high molecular weight polyethylene glycols and the like. The solid dosage forms of tablets, dragees, lozenges, capsules, pastilles, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar Form may also be employed as fillers in soft and hard-filled gelatin capsules using such excipients as lactose or milk sugar as well as high molecular weight polyethylene glycols and the like.

Provided compounds can also be in micro-encapsulated form with one or more excipients as noted above. The solid dosage forms of tablets, dragees, lozenges, capsules, pastilles, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions provided herein may be formulated for oral administration. Such formulations may be administered with or without food. The compositions are formulated in unit dosage forma for ease of administration and uniformity of dosage. The expression “unit dosage form” as used herein refers to a physically discrete unit of agent appropriate for the patient to be treated. It will be understood, however, that the total daily usage of the compositions of the present disclosure will be decided by the attending physician within the scope of sound medical judgment.

The amount of the compound of Formula I that may be included in a single dosage form will vary depending upon the patient to be treated (e.g. child vs adult, etc.). Provided compositions may be formulated such that a total daily dosage of, for instance, between 0.01 and 20 mg/kg body weight/day of the compound can be administered to a patient receiving these compositions. Single dose compositions may contain such an amount, or the total daily dose may be divided in multiple dosage forms to be taken, for instance, one, two or three times a day. For instance, a single dose may include between 5 and 500 mg of the active ingredient, or between 20 and 200 mg. Treatment regimens may comprise administration to a patient a total amount of from about 10 mg to about 1000 mg of the compound(s) of the present description per day in a single dose or divided in multiple doses.

It will be understood, that the total daily dose of the compound of Formula I will be decided by the attending physician within the scope of sound medical judgment. For instance, a specific dosage or treatment regimen for any particular patient will depend upon a variety of factors, including age, body weight, general health, sex, diet, time of administration, rate of excretion, drug combination, the judgment of the treating physician, and the severity of the symptoms associated with the disease or disorder.

Depending upon the disease or disorder to be treated, additional therapeutic agents may also be present in the compositions of this disclosure or co-administered separately. Non-limiting examples of additional therapeutic agents which could be used in combination with the compound of Formula I include antidiabetic agents, cholesterol-lowering agents, anti-inflammatory agents, antimicrobial agents, matrix metalloproteinase inhibitors, lipoxygenase inhibitors, cytokine antagonists, immunosuppressants, anti-cancer agents, anti-viral agents, cytokines, growth factors, immunomodulators, prostaglandins, or anti-vascular hyperproliferation compound. The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF)), and agents used to attenuate an adverse effect of the present compound or of a co-administered ingredient.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

In some embodiments, the therapeutically effective amount of a compound as defined herein, or a pharmaceutically acceptable salt thereof, can be administered to a patient alone or admixed with a pharmaceutically acceptable carrier.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present description that, upon administration to a recipient, is capable of providing, either directly or indirectly, a compound of the present description or an inhibitory active metabolite or residue thereof.

### EXPERIMENTS AND EXAMPLES

### Materials and Instruments

Polarized light microscopic (PLM) pictures were captured on a Nikon™ DS-Fi2 upright microscope at room temperature.

Powder X-ray Diffraction (XRPD) was performed with a Panalytical™ XPert Powder XRPD on a Si zero-background. The XRPD parameters used were as follows:

| | Parameters for Reflection Mode |
|---|---|
| X-Ray wavelength | Cu, kα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0131 |
| Scan speed (°/s) | 0.16 |
| Experiment time | ~4 min |

Differential Scanning calorimetry (DSC) was performed on a TA Q2000 DSC from TA Instruments™. The DSC method was as follows: ramp from RT to desired temperature at a heating rate of 10° C./min using N₂ as the purge gas, with crimped pan having a pin hole in the lid. The DSC parameters were as follows:

| Parameters | DSC |
|---|---|
| Pan Type | Aluminum pan, closed |
| Temperature | RT-300° C. |
| Ramp rate | 10° C./min |

Thermogravimetric Analysis (TGA) was performed on a TA Q500 TGA from TA Instruments. The TGA method was as follows: ramp from RT to desired temperature (300° C.) at a heating rate of 10° C./min using N₂ as the purge gas. The TGA parameters were as follows:

| Parameters | TGA | |
|---|---|---|
| Pan Type | Platinum plate, open | |
| Temperature | 300° | C. |
| Ramp rate | 10° | C./min |
| Purge gas | N₂ | |
| Sample purge flow | 15 | mL/min |
| Balance purge flow | 25 | mL/min |

Karl Fisher (KF) was performed on a Mettler Toledo™ volumetric KF titrator, to determine the water content in the samples.

The solvent abbreviations are as follows:

| Abbre- | | Abbre- | |
|---|---|---|---|
| viation | Solvent | viation | Solvent |
| MeOH | Methanol | THF | Tetrahydrofuran |
| EtOH | Ethanol | 2-MeTHF | 2-Methyltetrahydrofuran |
| IPA | Isopropyl alcohol | DCM | Dichloromethane |
| MIBK | 4-Methyl-2-pentanone | ACN | Acetonitrile |
| EtOAc | Ethyl acetate | DMSO | Dimethylsulfoxide |
| IPAc | Isopropyl acetate | DMF | Dimethylformamide |
| MTBE | Methyl tert-butyl ether | MEK | Methyl ethyl ketone |

### Summary of the Experimental Results

A polymorph screening of the compound of Formula I was performed, to identify the most stable crystalline form.

A first crystalline form of the compound of Formula I was obtained through the method described in Example 2. This crystalline form was characterized by X-Ray Powder Diffraction (XRPD), Polarized Light Microscopy (PLM), Thermo-Gravimetric Analysis (TGA) and Differential Scanning calorimetry (DSC). The characterization results indicated that the starting material was an anhydrous crystalline material with rod-like particles. DSC showed a single melting endotherm with an onset of 152.2° C. This crystalline Form was designated as Form A.

Starting with Form A, a polymorph screening was performed under 100 different conditions, using methods of slurry conversion at room-temperature (RT) and 60° C., temperature cycling, anti-solvent addition, slow cooling, slow evaporation, polymer induced crystallization, crash cooling, bulky solvent slurry, and reverse anti-solvent addition. The screening produced 17 crystalline forms (Forms A, B, C, D, E, F, G, H, I, J, K, L, M, N, O, P, and Q):
Forms A, C, D, E, N, and Q were found to be anhydrates;
Forms A, D and E were found to products of desolvation of the labile solvates Forms J, H, and P, respectively;
Forms C, N, and Q were found to be true anhydrates where no change in XRPD pattern was observed between wet and dry cake samples;
Form K was found to be a hemi-hydrate (1:0.5 API:H₂O ratio);
Forms B, F, L, M, and O were found to be solvates; and
Wet cake XRPD analysis of the solvates produced 2 new crystalline patterns, Forms I and G, which upon ambient drying produced Forms B and F, respectively.

A thermodynamic assessment of the six anhydrates (Forms A, C, D, E, N, Q) was performed in a non-solvating solvent, n-butanol to determine the most stable phase at RT and 50° C. Both experiments showed full conversion to Form C, indicating that Form C is the thermodynamically most stable anhydrous form under those conditions.

### Example 1: Polymorph Screening

Polymorph screening experiments were performed using different solution crystallization or solid phase transition methods starting with Form A. The methods used and crystal forms identified are summarized in Table 1-1

TABLE 1-1
Summary of polymorph screening experiments

| | No. of | |
|---|---|---|
| Method | Experiment | Crystal Form Identified |
| Anti-solvent addition | 20 | Type A, C, E, G, J, N, P |
| Slow evaporation | 10 | Type A, J, O, |
| Crash Cooling | 4 | Type K, M, L |
| Slurry at RT | 20 | Type A, B, E, F, G, I, N, P |
| Slurry at High Temperature | 10 | Type B, C, D, H, I, K, N, |
| Bulky Solvent Slurry | 6 | Type N, Q |
| Slow Cooling | 5 | Type D, H, I, K, L |
| Polymer induced | 5 | Type A, K |
| crystallization | | |
| Temperature Cycling | 10 | Type A, B, C, D, E, F, H, I, P |
| Reverse Anti-Solvent | 10 | Type A, J, N |
| Total | 100 | 17 |

### Anti-Solvent Addition

About 20-50 mg of Form A was dissolved in appropriate solvent to obtain a clear solution. The solution was magnetically stirred followed by addition of 0.2 mL anti-solvent stepwise until precipitate appeared or the total amount of anti-solvent reached 15.0 mL. The obtained precipitate was isolated for XRPD analysis. A total of 20 experiments were conducted. Results in Table 1-2 showed that Form A, C, E, G, J, N, and P were obtained.

TABLE 1-2
Summary of anti-solvent addition experiments

| Solvent | Anti-solvent | Solid Form |
|---|---|---|
| ACN | H₂O | A/J |
| EtOH | H₂O | C |
| Acetone | H₂O | A |
| 2-MeTHF | H₂O | E/P |
| DMF | H₂O | Amorphous |
| THF | H₂O | Amorphous |
| Acetone | n-Heptane | A/J |
| Chloroform | n-Heptane | N |
| DCM | n-Heptane | N |
| EtOAc | n-Heptane | N |
| MEK | n-Heptane | N |
| MIBK | n-Heptane | N |
| IPAc | n-Heptane | N |
| MEK | MTBE | Amorphous |
| ACN | MTBE | A/J |
| 2-MeTHF | MTBE | Amorphous |
| Acetone | MTBE | Amorphous |
| THF | MTBE | Amorphous |
| EtOAc | MTBE | Amorphous |
| 1,4-Dioxane | MTBE | G/F |

### Slow Evaporation

Slow evaporation experiments were performed under 10 conditions. About 20 mg of Form A was dissolved in 1.0 mL of solvent in a 3-mL glass vial. The visually clear solutions were allowed a slow evaporation at RT. The solids were isolated for XRPD analysis. The results summarized in Table 1-3 indicated that Form A, E, F, G, J, and K were obtained.

TABLE 1-3
Summary of slow evaporation experiments

| Solvent | Solid Form |
|---|---|
| DCM | A/J |
| CHCl3 | A/J |
| MEK | A/J |
| IPAc | Amorphous |
| THF | Amorphous |
| EtOAc | Amorphous |
| ACN | A/J |
| Dioxane | G/F |
| EtOH | K |
| DMF | E |

### Crash Cooling

About 25-50 mg of Form A dissolved in 0.2-0.6 mL solvent to get a suspension. The suspension was then heated to 60° C. using magnetic stirring and equilibrated for two hours and filtered using a PTFE membrane (pore size of 0.20 μm). Filtrates were immediately transferred to an environment of 5° C. A total of 4 experiments were conducted. Results in Table 1-4 showed that Form E, K, L, M, and P were obtained.

TABLE 1-4
Summary of crash cooling experiments

| Solvent (v:v) | Solid Form |
|---|---|
| EtOH | K |
| IPAc | M |
| Anisole | L |
| Toluene | E/P |

### Slurry at RT

Slurry conversion experiments were conducted at RT in 20 solvent systems. About 15-20 mg of Form A was suspended in 0.5 mL-1.0 mL of solvent at RT. The remaining solids were isolated for XRPD analysis. Result summarized in Table 1-5 indicated that Form A, B, E, F, G, I, N, and P were obtained.

TABLE 1-5
Summary of slurry conversion experiments at RT

| Solvent (v/v) | Solid Form |
|---|---|
| MIBK/IPA (1:9) | B/I |
| EtOH/IPA (1:9) | B/I |
| DMSO/IPA (1:9) | Amorphous |
| ACN/IPA (1:9) | B/I |
| 2-MeTHF/MTBE (1:9) | E/P |
| Acetone/MTBE (1:9) | E/P |
| IPAc/MTBE (1:9) | E/P |
| DCM/MTBE (1:9) | A/J |
| CHCl3/Heptane (1:9) | N |
| Dioxane/Heptane (1:9) | N |
| Toluene | A/J |
| Anisole | E/P |
| IPA | B/I |
| MTBE | E/P |
| DMF | Gel |
| H2O | Amorphous |
| THF/H2O (Aw = 0.2, 0.92/0.08) | F/G |
| THF/H2O (Aw = 0.4, 0.83/0.17) | F/G |
| THF/H2O (Aw = 0.6, 0.73/0.27) | F/G |
| THF/H2O (Aw = 0.8, 0.6/0.4) | Amorphous |

### Slurry at 60° C.

Slurry conversion experiments were conducted at 60° C. in 10 solvent systems. About ˜25-75 mg of Form A was suspended in 0.5 mL of solvent at 60° C. for 3 days. The remaining solids were isolated for XRPD analysis. Result summarized in Table 1-6 indicated that Form B, C, D, H, I, K, and N were obtained from all experiments.

TABLE 1-6
Summary of slurry conversion experiments at 60° C.

| Solvent (v/v) | Solid Form |
|---|---|
| Toluene | D/H |
| Water | C |
| Heptane | N |
| IPA | B/I |
| DMF/Toluene (1:9) | Amorphous |
| MIBK/Heptane (1:4) | C |
| MEK/Heptane (1:4) | N |
| EtOH/Toluene (1:5) | K |
| EtOAC/Toluene (1:5) | D/H |
| 2Me-THF/IPA (1:4) | B/I |

### Slurry in Bulky Solvents

Slurry conversion experiments were conducted at 60° C. and 100° C. in 6 different bulky solvent systems. About ˜25-75 mg of Form A was suspended in 0.5 mL of bulky solvent at the corresponding temperature and magnetically stirred. The remaining solids were isolated for XRPD analysis. Result summarized in Table 1-7 indicated that Form N and Q were obtained from all experiments.

TABLE 1-7
Summary of slurry conversion experiments in bulky solvents

| Solvent (v/v) | Temperature | | Solid Form |
|---|---|---|---|
| Iso-Octane | 60° | C. | N |
| Iso-Butanol | 100° | C. | Q |
| Propylene Glycol | 100° | C. | Q |
| Ethylene Glycol | 100° | C. | Q |
| n-Butanol | 100° | C. | Q |
| n-Pentanol | 100° | C. | Q |

### Slow Cooling

About 25-50 mg of Form A was dissolved in 0.2-0.6 mL solvent to get a suspension. The suspension was then heated to 50° C. using magnetic stirring and equilibrated for two hours and filtered using a PTFE membrane (pore size of 0.20 μm). Filtrates were slowly cooled down to 5° C. at a rate of 0.1° C./min. A total of 5 experiments were conducted. Results in Table 1-8 showed that Form D, H, K, and L were obtained.

TABLE 1-8
Summary of slow cooling experiments

| Solvent (v:v) | Solid Form |
|---|---|
| EtOH | K |
| IPAc | Amorphous |
| Anisole | L |
| Toluene | D/H |
| n-Heptane | No Solids |

### Polymer Induced Crystallization

Polymer induced crystallization experiments were performed with two different polymers in 5 solvents. About 20 mg of Form A was dissolved in 0.5˜2.0 mL of appropriate solvent to obtain a clear solution in a 3-mL vial. About 2 mg of polymer mixture was added into 3-mL glass vial. All the samples were subjected to evaporate at RT to induce precipitation. The solids were isolated for XRPD analysis. Results summarized in Table 1-9 showed that Form A, J, and K were obtained.

TABLE 1-9
Summary of polymer induced crystallization experiments

| Solvent (v:v) | Polymer | Solid Form |
|---|---|---|
| H₂O | polyvinylchloride (PVC) | Amorphous |
| EtOH | polyvinylchloride (PVC) | K |
| DCM | polyvinylchloride (PVC) | A/J |
| EtOAc | sodium alginate (SA) | Amorphous |
| n-Heptane | sodium alginate (SA) | Low crystallinity |

### Temperature Cycling

Suspensions of Form A about 50-100 mg/ml in 0.5 ml of solvent were magnetically stirred and exposed to high (50° C.) and low (5° C.) temperature in cycles. Solids were isolated at day 1 and day 3 and analyzed by XRPD. A total of 10 experiments were conducted as summarized in Table 7-15 and Form A, B, C, D, E, F, G, H, I, J, and N were obtained.

TABLE 1-10
Summary of temperature cycling experiments
between 50° C. and 5° C.

| Solvent (v/v) | Solid Form Day 1 | Solid Form Day 3 |
|---|---|---|
| n-Heptane | A/J | N |
| IPA | B/I | B/I |
| H₂O | C | C |
| Toluene | D/H | D/H |
| IPAc/MTBE (1:3) | E/P | E/P |
| Acetone/Heptane (1:4) | A/J | A/J |
| CHCl₃/Heptane (1:3) | N | N |
| THF/H₂O (1:3) | F/G | F/G |
| 2-MeTHF/H₂O (1:4) | Amorphous | Amorphous |
| 2-MeTHF/n-Heptane | A/J | N |

### Reverse Anti-Solvent Addition

About 20-50 mg of Form A was dissolved in appropriate solvent to obtain a clear solution. The solution was then added dropwise into the corresponding anti-solvent with magnetic stirring. The obtained precipitate was isolated for XRPD analysis. A total of 10 experiments were conducted. Results in Table 1-11 showed that Form A, J, and N were obtained.

TABLE 1-11
Summary of reverse anti-solvent addition experiments

| Solvent | Anti-solvent | Solid Form |
|---|---|---|
| ACN | H₂O | Amorphous |
| EtOH | H₂O | Amorphous |
| MeOH | H₂O | Amorphous |
| DMF | H₂O | Amorphous |
| Dioxane | H₂O | Amorphous |
| THF | H₂O | Amorphous |
| Acetone | n-Heptane | A/J |
| Chloroform | n-Heptane | N (gel) |
| DCM | n-Heptane | N (gel) |
| EtOAc | n-Heptane | N |

XRPD of the isolatable forms is can be seen in the Figures. The characterization results are summarized in Table 1. A phase map of the six anhydrates is shown at FIG. 1A. A phase interconversion map of all solvates and hydrates is shown at FIG. 1B.

TABLE 1
Characterization of crystalline forms of the compound of Formula I.

| | | | | DSC | Form | Stoichiometry |
|---|---|---|---|---|---|---|
| | | | | (onset or | Identification | API:Solvent |
| Form/ | Preparation | TGA | KF | peak* | based on TGA | based on TGA & |
| Sample ID | condition | (weight %) | (H₂O %) | ° C.) | & KF | KF |
| Form A | Obtained from | 0.01 | NA | 152.2 | Anhydrate | NA |
| | synthesis (see | | | | | |
| | Example 2) | | | | | |
| Form B | Temperature | 5.34 | 0.5 | 126.4 | Solvate | 1:0.55 |
| | Cycling in IPA | | | | | |
| | (Air Dry XRPD) | | | | | |
| Form C | Temperature cycling | 0.32 | NA | 157.1 | Anhydrate | NA |
| | in H₂O or | | | | | |
| | Anti-solvent addition | | | | | |
| | EtOH/H₂O | | | | | |
| | (XRPD wet solids) | | | | | |
| Form D | Temperature | 0.07 | NA | 155.9 | Anhydrate | NA |
| | Cycling in Toluene | | | | | |
| | (Air Dry XRPD) | | | | | |
| Form E | Temperature | 0.43 | NA | 120.9, | Anhydrate | NA |
| | Cycling in | | | 146.1 | | |
| | IPAc/MTBE (1:3) | | | | | |
| | (Air Dry) | | | | | |
| Form F | RT slurry in | 1.85 | 0.6 | 119.5 | Partially | 1:0.15 (THF) |
| | THF/H2O | | | | desolvated | |
| | (0.92/0.08; | | | | | |
| | Aw = 0.2) (Air Dry | | | | | |
| | XRPD) | | | | | |
| Form G | RT slurry in | NA | NA | NA | Labile | NA |
| | THF/H2O | | | | Solvate | |
| | (0.92/0.08; | | | | | |
| | Aw = 0.2) (Wet | | | | | |
| | XRPD) | | | | | |
| Form H | Temperature | NA | NA | NA | Labile | NA |
| | Cycling in Toluene | | | | Solvate | |
| | (Wet XRPD) | | | | | |
| Form I | Temperature | NA | NA | NA | Labile | NA |
| | Cycling in IPA | | | | Solvate | |
| | (Wet XRPD) | | | | | |
| Form J | Anti-Solvent | NA | NA | NA | Labile | NA |
| | Addition | | | | Solvate | |
| | ACN/H2O | | | | | |
| | (Wet XRPD) | | | | | |
| Form K | Crash Cooling | 1.35 | 1.4 | 53.6, | Hemi- | 1:0.47 |
| | in EtOH | | | 106.3 | Hydrate | |
| Form L | Crash Cooling | 4.56 | 0.21 | 126.4 | Solvate | 1:0.26 |
| | in | | | | | |
| | Anisole | | | | | |
| Form M | Crash Cooling | 13.33 | 0.17 | 108.9, | Solvate | 1:0.89 |
| | in IPAc | | | 116.7*, | | |
| | | | | 128.2* | | |
| Form N | Temperature | 0.46 | NA | 142.0 | Anhydrate | NA |
| | Cycling in | | | | | |
| | CHCl3/n-Heptane | | | | | |
| | (1:3) | | | | | |
| Form O | Slow | 5.11 | Minimal | 107.4 | Very Likely | 1:0.99 |
| | Evaporation | | solids | | Solvate | |
| | in MeOH | | produced | | | |
| Form P | Temperature | NA | NA | NA | Labile | NA |
| | Cycling in | | | | Solvate | |
| | IPAc/MTBE (1:3) | | | | | |
| | (Air Dry) | | | | | |
| Form Q | 100 0 C. slurry | 0.63 | NA | 101.4 | Anhydrate | NA |
| | in n-Butanol | | | | | |

### Example 2: Form A and Form J

Form A was obtained via the following synthesis step:

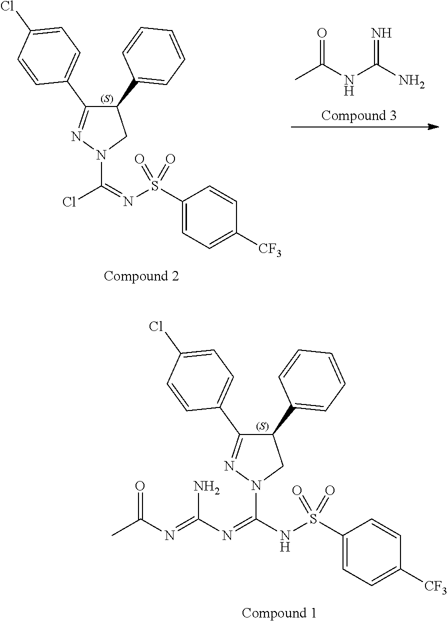

1) 7.75 L of DMF was pumped into a 50 L enamel jacket kettle by reduced pressure agitated.
2) 1.58 kg of Compound 2 was added to the solution at 25-30° C. in one portion.
3) 0.595 kg of Compound 3 was added to the solution at 25-30° C. in one portion.
4) The reaction mixture was stirred at 20-30° C. for 0.5 h.
5) 1.49 kg of TEA was added to the solution at 25-30° C. under nitrogen.
6) The reaction mixture was stirred at 20-30° C. for 1.0 h.
7) The reaction was monitored by HPLC until completion.
8) After completion, the reaction mixture was slowly added into 31 L of water and stirred at 10-15° C. for 30 min.
9) The reaction mixture was then filtered, and the filter cake was washed with water (2000 mL). The washed filter cake was dissolved in DCM (4000 mL) and water (1000 mL) was added.
10) The organic layers were washed with brine (3000 mL), dried over Na₂SO₄, filtered and concentrated under reduced pressure to give a crude compound having a purity of 93.1% (by HPLC).
11) The crude compound of Formula I was purified by column chromatography (SiO₂, Petroleum ether/Ethyl acetate=3/1 to 2/1, TLC: PE:EA=1:1, p1:Rf=0.5).
12) The collected fractions were concentrated under reduced pressure to give a residue.
13) The residue was triturated with DCM (5.0 L) and n-heptane (5.0 L) at 30° C. for 1.0 h.
14) The mixture was then filtered, and the filter cake was dried under reduced pressure at 45° C.
15) Crystalline compound of Formula I, Form A (1.48 kg, 85% yield, purity 99.8%) was obtained as a white solid, which was confirmed by HPLC, LCMS and H/FNMR.

Form A was characterized by XRPD (FIG. 2A) and PLM. Form A was shown to be crystalline and possess small rod-like particles with agglomeration.

Thermal analysis conducted on Form A showed a weight loss of 0.01% up to 200° C., and a single endothermic peak with an onset of 152.2° C. and a peak temperature of 162.3° C. as displayed in FIG. 2B.

Form A obtained from anti-solvent addition in ACN/H₂O was further investigated by analyzing the wet solids by XRPD. The sample was covered with 3511 Kapton film to minimize the solvent evaporation from the wet cake. A unique pattern, different from Form A was observed indicating a labile solvate phase forms in this solvent system. This crystalline form was designated as Form J. Upon ambient drying, Form J converted to Form A. XRPD overlay of Form A and Form J is shown in FIG. 2C.

Approximate solubility of Form A was determined in 20 single solvents at RT, respectively. Approximately 2 mg of Form A was added into a 3-mL glass vial. Corresponding solvents were added stepwise (50 μL-50 μL-200 μL-700 μL) into the vials until the solids were dissolved visually or a total volume of 1 mL was reached. The ranges of approximate solubility were calculated according to the mass of sample, volume of solvent and observation. The results summarized in Table 2 were used to guide the solvent selection in polymorph screening.

Form A had high solubility (>10 mg/mL) in most solvents including MeOH, EtOH, Acetone, MIBK, MEK, EtOAc, IPAC, THF, 2-MeTHF, 1,4-Dioxane, Anisole, ACN, CHCl₃, DMSO, and DCM. Moderate solubility (2.0<S<10.0) was observed in Toluene. Low solubility (<3.0 mg/mL) was observed in IPA, MTBE, n-Heptane, and H₂O.

TABLE 2
Solubility of the compound of Formula I, Form A

| Solvent | Solubility (mg/mL) | Solvent | Solubility (mg/mL) |
|---|---|---|---|
| MeOH | S > 36.0 | Dioxane | S > 64.0 |
| EtOH | 18.0 < S < 36.0 | MTBE | S < 2.4 |
| IPA | S < 3.1 | Anisole | 7.7 < S < 23.0 |
| Acetone | S > 42.0 | n-heptane | S < 2.7 |
| MIBK | S > 44.0 | ACN | S > 70.0 |
| MEK | S > 40.0 | Water | S < 2.9 |
| EtOAc | S > 40.0 | Toluene | 2.8 < S < 9.3 |
| IPAC | 33.0 < S < 66.0 | CHCl3 | S > 40.0 |
| THF | S > 34.0 | DMSO | S > 52.0 |
| 2-MeTHF | S > 54.0 | DCM | S > 42.0 |

### Example 3: Form B and Form I

Form B was obtained by temperature cycling in IPA. The formation of Form B was also observed in IPA slurry at RT and 60° C. Form B was further characterized by analyzing the wet cake by XRPD. The sample on the XRPD sample holder was covered by 3511 Kapton Film to minimize the solvent evaporation from the sample. A unique pattern, different from Form A and Form B, was observed, and was designated as Form I. Upon ambient drying, Form I converted to Form B, indicating that Form I is a labile solvate. Comparison of Form A, Form B, and Form I XRPD patterns is displayed in FIG. 3A. PLM showed small irregularly shaped birefringent particles. Thermal analysis in FIG. 3B showed a weight loss of 5.34% up to 174.0° C. by TGA, and a broad endotherm with an onset of 126.4° C. and a peak temperature of 136.3° C. by DSC. KF analysis of Form B and showed 0.5% water content. Comparing the KF and TGA results, it was concluded that Form B is an IPA solvate, more specifically, a hemi-solvate determined by calculating the API:Solvent stoichiometry from the TGA weight loss to be 1:0.6.

### Example 4: Form C

Form C was obtained by temperature cycling in H₂O from 60° C. to 25° C. The formation of Form C was also observed in anti-solvent addition and 60° C. screening experiments. Comparison of Form A and Form C XRPD patterns is shown in FIG. 4A. PLM image of Form C showed small irregularly shaped birefringent particles. Thermal analysis in FIG. 4B showed a weight loss 0.32% up to 175.0° C. and a single endotherm with an onset of 157.1° C. and a peak temperature of 167.9° C. The minimal weight loss observed indicated an anhydrous phase.

### Example 5: Form D and Form H

Form D was obtained by temperature cycling in toluene. The formation of Form D was also observed in 60° C. slurry and slow cooling using toluene. Form D was further characterized by analyzing the wet solids by XRPD. The sample on the XRPD sample holder was covered by 3511 Kapton Film to minimize the solvent evaporation from the sample. A unique pattern, different from Form A and Form D, was observed indicating a labile solvate phase, and was designated as Form H. Upon ambient drying, Form H converted to Form D. Comparison of Form A, Form D, and Form H XRPD patterns is displayed in FIG. 5A. PLM image of Form D showed small irregular needles with agglomeration. Thermal analysis in FIG. 5B showed a weight loss 0.07% up to 180.0° C. and a single endotherm with an onset of 155.9° C. and an enthalpy of 23.5 J/g, and a peak temperature of 163.9° C. The minimal weight loss observed indicated an anhydrous phase.

### Example 6: Form E and Form P

Form E was obtained by temperature cycling in IPAc/MTBE (1:3 v/v). The formation of Form E was also observed in anti-solvent addition, crash cooling, and slurry at RT experiments using MTBE, 2-MeTHF, or toluene. Form E was further characterized by analyzing the wet solids by XRPD. The sample on the XRPD sample holder was covered by 3511 Kapton Film to minimize the solvent evaporation from the sample. A unique pattern, different from Form A and Form E, was observed indicating a labile solvate phase, and was designated as Form P. Upon ambient drying, Form P converted to Form E. Comparison of Form A, Form E, and Form P XRPD patterns is displayed in FIG. 6A. PLM image of Form E showed small irregularly shaped birefringent particles. Thermal analysis in FIG. 6B showed a weight loss 0.31% up to 177.9° C. and two endotherms, with onsets of 120.6° C. and 146.0° C. and peak temperatures of 129.5° C. and 154.6° C. respectively. The minimal weight loss observed indicated an anhydrous phase.

The first small endotherm was further investigated to determine if a conversion to another form occurred upon heating past the first small endotherm and before the final melting endotherm, and then cooling back down to room temperature. Cycle DSC shown in FIG. 6C was performed by heating to 135° C. in cycle 1, cooling to RT in cycle 2, then heating to 300° C. (past the final melt) in cycle 3. In cycle 2, the endothermic event that occurred in cycle 1 (onset 122.1° C.) was observed upon cooling indicating that it is a reversible event. In the final cycle, the first endotherm is again observed. This indicates Form E converts to a high temperature melting point that cannot be isolated, because it converts back to Form E upon cooling. This is an indication that Form E and the high temperature melting form are enantiotropically related, with Form E being more stable at temperatures <122° C. The melting endotherm at 147.4° C. does not match the melting of any of the other forms observed in the screening providing additional evidence that a new form exists at the elevated temperature. XRPD of this new form can be obtained by in situ hot stage XRPD.

### Example 7: Form F and Form G

Form F was obtained by temperature cycling in THF/H₂O (0.92/0.08 v/v A_{w}=0.2). The formation of Form F was also observed in RT slurry in THF/H₂O at A_{w}=0.4 and 0.6, and in temperature cycling in THF/H₂O (1:3). Form F was further characterized by analyzing the wet solids by XRPD. The sample on the XRPD sample holder was covered by 3511 Kapton film to minimize the solvent evaporation. A unique pattern, different from Form A and Form F, was observed indicating a labile solvate phase, which was designated as Form G. Upon ambient drying, Form G converted to Form F. Comparison of Form A, Form F, and Form G XRPD patterns is displayed in FIG. 7A. PLM image showed small irregularly shaped birefringent particles. Thermal analysis in FIG. 7B showed a weight loss of 1.85% up to 165.0° C. by TGA, and a broad endotherm with an onset of 119.5° C. and a peak temperature of 132.4° C. KF was obtained of Form F and resulted in a water content of 0.6%. Comparing the KF and TGA results, it was concluded that Form F is likely a THF solvate.

### Example 8: Form K

Form K was obtained by crash cooling from EtOH solution. The formation of Form K was also observed in slow evaporation, 60° C. slurry, slow cooling, and polymer induced crystallization using EtOH. Comparison of Form A and Form K XRPD patterns is displayed in FIG. 8A. PLM image showed small irregularly shaped birefringent single particles and agglomerates. Thermal analysis in FIG. 8B showed a weight loss of 1.35% up to 160.0° C. by TGA, and two endothermic events with onsets of 53.6° C. and 106.3° C. and peak temperatures at 68.7° C. and 125.9° C. respectively. Form K was heated to 90° C. and cooling back down. XRPD of the resulting material showed a change in compared patterns as shown in FIG. 8C due to the driving off of the water by heating resulting in the dehydrated phase. The water content of the solids was determined by KF to be 1.4%. The weight loss in TGA and water content obtained by KF were complimentary and Form K was concluded to be a hemi-hydrate.

### Example 9: Form L

Form L was obtained by crash cooling of anisole solution. The formation of Form L was also observed in slow cooling experiments using anisole. Comparison of Form A and Form L XRPD patterns is displayed in FIG. 9A. PLM image showed small irregularly shaped birefringent primary particles and agglomerates. Thermal analysis in FIG. 9B showed a weight loss of 4.56% up to 165.0° C. by TGA, and a single endothermic event with an onset of 126.4° C. and a peak temperature of 133.8° C. in the DSC curve that occurs after the solvent is driven. KF was obtained of Form L and resulted in a water content of 0.2%. Comparing the KF and TGA results, it was concluded that Form L is a partially desolvated anisole solvate as the stoichiometry of API:Anisole is 0:0.26.

### Example 10: Form M

Form M was obtained by crash cooling of IPAc solution. Comparison of Form A and Form M XRPD patterns is displayed in FIG. 10A. PLM image showed small irregular shaped birefringent particles. Thermal analysis in FIG. 10B showed a weight loss of 13.33% up to 165.0° C. by TGA, and 2 overlapping endothermic events in the DSC curve with an onset of 109° C., and peak temperatures at 116.7° C. and 128.2° C. that occur concurrently with the TGA weight loss step. KF was obtained of Form M and resulted in a water content of 0.2%. Comparing the KF and TGA results, it was concluded that Form M is an IPAc solvate, more specifically, a mono-solvate determined by calculating the API:Solvent stoichiometry from TGA weight loss.

### Example 11: Form N

Form N was obtained by temperature cycling n CHCl₃/n-Heptane (1:3 v/v). The formation of Form N was also observed in anti-solvent addition, RT and 60° C. slurry, bulky solvent slurry, and reverse anti-solvent addition experiments using n-Heptane. Comparison of Form A and Form N patterns is displayed in FIG. 11A. PLM image of Form N showed small irregularly birefringent particles. Thermal analysis in FIG. 11B showed a weight loss 0.46% up to 179.0° C. and a single endotherm with an onset of 141.9° C. and a peak temperature of 152.1° C. The minimal weight loss observed indicated an anhydrous phase.

### Example 12: Form O

Form O was obtained only by slow evaporation in MeOH. Comparison of Form A and Form O XRPD patterns is displayed in FIG. 12A. PLM image showed crystals with a large plate-like morphology. Thermal analysis in FIG. 12B showed a weight loss of 5.11% up to 160.0° by TGA, and a broad endotherm with an onset of 107.4° C. and a peak temperature of 110.6° C. Due to the method in which Form O was produced, minimal solids were isolated. Scaling up of Form O was attempted by cooling crystallization and using the remaining solids as seeds. This was unsuccessful and KF was not performed for this Form. However, calculating the stoichiometry of API:MeOH from the TGA weight loss shows 1:0.99 providing some evidence that Form O is a MeOH mono-solvate.

### Example 13: Form Q

Form Q was obtained by slurrying Form A in n-Butanol at 100° C. The formation of Form Q was also observed in 100° C. bulky solvent slurry using iso-butanol, propylene glycol, ethylene glycol, and n-pentanol. Comparison of Form A and Form Q XRPD patterns is displayed in FIG. 13A. PLM image of Form Q showed very small irregularly shaped birefringent particles. Thermal analysis in FIG. 13B showed a weight loss 0.63% up to 160.0° C. and a single broad endotherm with an onset of 101.4° C. and a peak temperature of 130.1° C. The minimal weight loss observed indicated an anhydrous phase.

### Example 14: Slurry Competition Experiments

### Anhydrates:

A total of six anhydrates, Forms A, C, D, E, N, and Q were observed. A thermodynamic assessment of all anhydrates was performed by slurrying all anhydrates in equal mass ratios in a non-solvating solvent, n-Butanol, at RT and 50° C. At both RT and 50° C., conversion of all anhydrates to Form C was observed indicating that it is the most stable anhydrate.

### Hydrates:

One hydrate (Form K) was observed and found to be a hemi-hydrate. A thermodynamic assessment of Form K was performed by creating a slurry of Form K and Form C (the most stable anhydrate) in equal mass ratios at various water activities using a non-solvating solvent. Initially, EtOH/H₂O was used, and results indicated that from the range, 0.1<A_{w}<0.9, Form K was more stable. However, in this experiment Form C was found to be more stable at A_{w}>0.9. It was suspected that EtOH may be forming a solvate that favors the formation of Form K because at higher water activities, the hydrate should be more thermodynamically stable. The values and results for obtaining the critical water activity follows the laws of thermodynamics and the results obtained from this experiment can be transferred to another solvent/H₂O system. Additional critical water activity experiments were performed in acetone/H₂O at A_{w}=0.5 and 0.9 using Form K and Form C. At both water activities, conversion to Form C was observed. These results indicate that Form K is not a stable hydrate and that the formation of Form K is influenced by the presence of EtOH.

### Example 15: Scale Up of Form C

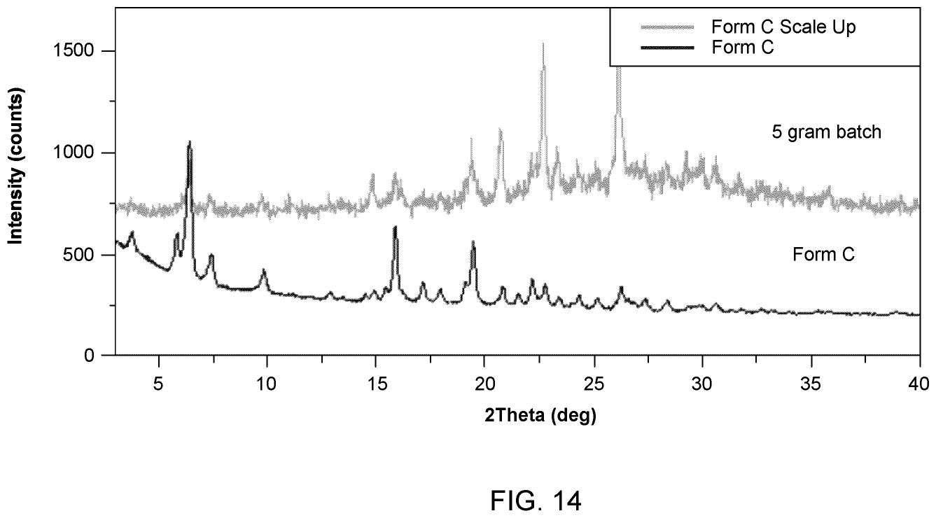

Form C was successfully scaled up to 5 grams. Starting with Form A, ˜5 grams were weighed and transferred to a 100 ml bottle. 50 mL of EtOH was added to create a suspension and the mixture was magnetically stirred at 65° C. Once a clear solution was obtained, the solution was slowly cooled to 50° C. Once the sample reached 50° C., H₂O was at added at a rate of ˜1 mL/min to the solution until a 1:1 ratio (v/v) of EtOH/H₂O was obtained. The sample was filtered, and the wet solids were evenly distributed onto a large weigh boat breaking apart any large clumps. A sample was extracted from the bulk solids to perform in house baseline characterization. XRPD of wet solids confirmed the formation of Form C as shown in FIG. 14. PLM image of the material showed needle-like morphology with some agglomeration. The extracted sample was vacuum dried at 40° C. for several hours, then analyzed by TGA/DSC. TGA results confirmed the anhydrous nature of Form C, and an increase in enthalpy (AH) was observed. The remaining bulk solids were then vacuum dried over a weekend.

### Example 16: Comparison of Simulated XRPD and Experimental XRPD Data

Single-crystal data of a compound of Formula I is disclosed in Liu et al., Functional Selectivity of a Biased Cannabinoid-1 Receptor (CB₁R) Antagonist, ACS Pharmacology & Translational Science, 2021, which is hereby incorporated by reference in its entirety.

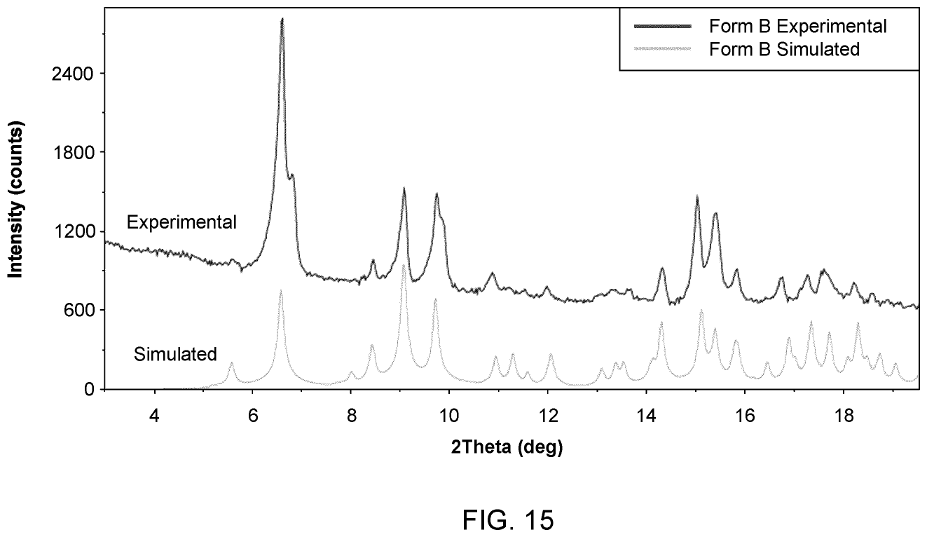

This single crystal data was matched to experimental XRPD data through simulation, and was shown to correspond to Form B. An overlay of the simulated XRPD data and of the experimental XRPD data of Form B is shown at FIG. 15.

The simulated XRPD pattern shows a strong match with the experimentally obtained pattern for Form B at low angles with minor adjustments (0.1° 20). There is noticeable drift across the peak locations across the two spectra, but the peak intensity ratios and relative locations when considered as groups is an excellent match. The single-crystal data was noted in the CIF to have been collected at 100 K while the XRPD data was collected at ambient conditions. The displacement on the x-axis is due to thermal contraction and expansion and is to be expected with a temperature disparity this great. It is significant that the peak patterns retain their similarity to the simulation so well, inferring that the temperature change causes a near-uniform shift in the unit cell parameters, and indicates that there is no polymorphic transition across the temperature range. In addition to the crystallographic evidence, the single-crystal data indicates that the crystal is a monosolvate of isopropyl alcohol and hemihydrate. While the ratios are not consistent with those reported for Form B, the solvent identified is consistent.

### Example 17: Other Experimental Conditions to Obtain Form C

Form C was obtained by temperature cycling in water:
3 g of the compound of Formula I, Form A were added to a flask;
10 vol water were added to the flask;
Temperature cycling was conducted from 60° C. to 25° C.;
The mixture was stirred for 4.5 h at 25° C.;
The precipitated crystalline solids were filtered and dried under nitrogen at 20-25° C. and identified as Form C by XRPD.

Form C was also obtained by water addition to an ethanol solution:
0.5 g of the compound of Formula I, Form A was dissolved in 6 vol EtOH at 60° C.;
2 vol water were added to the mixture;
0.5% seed Form C was added to the mixture;
The mixture was stirred for 1 h at 60° C.;
The mixture was then cooled to 25° C. over 4 h;
The mixture was then stirred for 8 h at 25° C.;
The precipitated crystalline solids were filtered and dried under nitrogen at 20-25° C. and identified as Form C by XRPD. The yield was of 81%.

Form C was also obtained by water addition to a methanol solution:
0.5 g of the compound of Formula I, Form A was dissolved in 5 vol MeOH at 60° C.;
0.2 vol water were added to the mixture;
0.5% seed Form C was added to the mixture;
The mixture was stirred for 1 h at 60° C.;
2.3 vol water were added to the mixture;
The mixture was stirred for 1 h at 60° C.;
The mixture was then cooled to 25° C. over 4 h;
The mixture was then stirred for 8 h at 25° C.;
The precipitated crystalline solids were filtered and dried under nitrogen at 20-25° C. and identified as Form C by XRPD. The yield was of 74%.

Form C was also obtained by water addition to a n-propanol solution:
0.5 g of the compound of Formula I, Form A was dissolved in 10 vol n-PrOH at 60° C.;
3.2 vol water were added to the mixture;
0.5% seed Form C was added to the mixture;
The mixture was stirred for 1 h at 53° C.;
1.8 vol water were added to the mixture;
The mixture was stirred for 1 h at 53° C.;
The mixture was then cooled to 25° C. over 4 h;
The mixture was then stirred for 13 h at 25° C.;
The precipitated crystalline solids were filtered and dried under nitrogen at 20-25° C. and identified as Form C by XRPD. The yield was of 82%.

Form C was also obtained in 5 g, 18 g and 115 g-scale crystallization experiments starting from by dissolving Form A in Ethanol at 40° C., adding water, cooling to 25° C., and collecting crystalline compound of Formula I, Form C (yield 92%).

### XRPD Peak Lists

| | | | | |
|---|---|---|---|---|
| Pos. | Height | FWHMLeft | d-spacing | Rel. Int. |
| [°2Th.] | [cts] | [°2Th.] | [Å] | [%] |

Form A:

| | | | | |
|---|---|---|---|---|
| 3.086090 | 183.494200 | 0.076752 | 28.62961 | 0.45 |
| 3.707256 | 169.922600 | 0.102336 | 23.83388 | 0.42 |
| 4.519086 | 154.441900 | 0.153504 | 19.55390 | 0.38 |
| 4.767668 | 95.149730 | 0.127920 | 18.53492 | 0.24 |
| 5.895159 | 40344.720000 | 0.204672 | 14.99228 | 100.00 |
| 9.390182 | 1958.212000 | 0.230256 | 9.41854 | 4.85 |
| 10.059180 | 151.920000 | 0.127920 | 8.79360 | 0.38 |
| 10.648700 | 479.452900 | 0.153504 | 8.30807 | 1.19 |
| 11.634810 | 54.030360 | 0.307008 | 7.60604 | 0.13 |
| 12.381600 | 137.328200 | 0.153504 | 7.14892 | 0.34 |
| 12.959010 | 1182.441000 | 0.255840 | 6.83165 | 2.93 |
| 14.120070 | 74.544290 | 0.153504 | 6.27241 | 0.18 |
| 14.384260 | 89.043460 | 0.076752 | 6.15779 | 0.22 |
| 15.362120 | 106.397700 | 0.102336 | 5.76796 | 0.26 |
| 15.886470 | 431.711300 | 0.179088 | 5.57874 | 1.07 |
| 16.167840 | 654.325000 | 0.153504 | 5.48228 | 1.62 |
| 16.959060 | 850.349500 | 0.255840 | 5.22825 | 2.11 |
| 17.395840 | 2363.185000 | 0.153504 | 5.09795 | 5.86 |
| 17.804300 | 1452.938000 | 0.153504 | 4.98190 | 3.60 |
| 18.353340 | 243.036700 | 0.179088 | 4.83409 | 0.60 |
| 18.616340 | 181.817200 | 0.102336 | 4.76639 | 0.45 |
| 19.504140 | 618.851400 | 0.179088 | 4.55139 | 1.53 |
| 19.853480 | 877.906900 | 0.204672 | 4.47208 | 2.18 |
| 20.248410 | 223.899100 | 0.127920 | 4.38574 | 0.55 |
| 21.097250 | 78.173740 | 0.102336 | 4.21116 | 0.19 |
| 21.822750 | 254.780300 | 0.307008 | 4.07277 | 0.63 |
| 22.916980 | 266.261600 | 0.102336 | 3.88073 | 0.66 |
| 23.997590 | 178.567200 | 0.102336 | 3.70837 | 0.44 |
| 24.344500 | 248.531300 | 0.102336 | 3.65631 | 0.62 |
| 25.001850 | 401.287800 | 0.204672 | 3.56164 | 0.99 |
| 25.368100 | 127.185200 | 0.102336 | 3.51105 | 0.32 |
| 25.661320 | 185.998300 | 0.102336 | 3.47159 | 0.46 |
| 26.780970 | 36.757570 | 0.153504 | 3.32894 | 0.09 |
| 27.523320 | 28.599570 | 0.307008 | 3.24082 | 0.07 |
| 28.202270 | 69.732460 | 0.307008 | 3.16432 | 0.17 |
| 29.155480 | 74.018680 | 0.204672 | 3.06300 | 0.18 |
| 29.812190 | 46.653190 | 0.076752 | 2.99701 | 0.12 |
| 30.211580 | 44.721800 | 0.204672 | 2.95829 | 0.11 |
| 33.151900 | 29.172220 | 0.409344 | 2.70233 | 0.07 |
| 33.555400 | 6.709808 | 0.076752 | 2.67075 | 0.02 |
| 34.322070 | 60.547740 | 0.358176 | 2.61283 | 0.15 |
| 35.097970 | 81.229270 | 0.204672 | 2.55683 | 0.20 |
| 35.834360 | 20.889450 | 0.076752 | 2.50596 | 0.05 |
| 36.803920 | 33.269680 | 0.307008 | 2.44214 | 0.08 |

Form B:

| | | | | |
|---|---|---|---|---|
| 3.081334 | 53.230170 | 0.076752 | 28.67379 | 0.34 |
| 3.249706 | 168.982500 | 0.076752 | 27.18853 | 1.07 |
| 4.759403 | 70.288630 | 0.409344 | 18.56709 | 0.45 |
| 5.590357 | 219.460400 | 0.076752 | 15.80900 | 1.39 |
| 6.595675 | 15774.250000 | 0.102336 | 13.40146 | 100.00 |
| 6.827613 | 3869.795000 | 0.102336 | 12.94672 | 24.53 |
| 8.450346 | 628.405000 | 0.102336 | 10.46383 | 3.98 |
| 9.089335 | 3421.267000 | 0.127920 | 9.72960 | 21.69 |
| 9.727601 | 3338.140000 | 0.102336 | 9.09259 | 21.16 |
| 9.884985 | 1988.280000 | 0.076752 | 8.94817 | 12.60 |
| 10.866150 | 524.915400 | 0.102336 | 8.14229 | 3.33 |
| 11.516460 | 130.023300 | 0.076752 | 7.68394 | 0.82 |
| 11.963940 | 258.657900 | 0.127920 | 7.39753 | 1.64 |
| 12.960680 | 103.954800 | 0.102336 | 6.83078 | 0.66 |
| 13.647040 | 240.041800 | 0.102336 | 6.48874 | 1.52 |
| 14.317250 | 882.790200 | 0.127920 | 6.18646 | 5.60 |
| 15.017310 | 3571.110000 | 0.127920 | 5.89961 | 22.64 |
| 15.403460 | 2891.514000 | 0.153504 | 5.75257 | 18.33 |
| 15.819520 | 797.909400 | 0.127920 | 5.60220 | 5.06 |
| 16.725450 | 527.029900 | 0.127920 | 5.30074 | 3.34 |
| 17.257070 | 565.594500 | 0.102336 | 5.13863 | 3.59 |
| 17.542590 | 615.927200 | 0.179088 | 5.05563 | 3.90 |
| 18.201460 | 424.163000 | 0.076752 | 4.87408 | 2.69 |
| 18.561580 | 200.214100 | 0.076752 | 4.78032 | 1.27 |
| 18.884760 | 75.524100 | 0.102336 | 4.69924 | 0.48 |
| 19.837210 | 1155.661000 | 0.127920 | 4.47571 | 7.33 |
| 20.669110 | 182.399200 | 0.127920 | 4.29741 | 1.16 |
| 21.106720 | 374.324600 | 0.153504 | 4.20929 | 2.37 |
| 21.900850 | 1810.287000 | 0.153504 | 4.05843 | 11.48 |
| 22.338730 | 73.347710 | 0.127920 | 3.97986 | 0.46 |
| 22.779130 | 6.581588 | 0.153504 | 3.90390 | 0.04 |
| 23.387450 | 692.068500 | 0.230256 | 3.80371 | 4.39 |
| 24.268100 | 146.559900 | 0.204672 | 3.66764 | 0.93 |
| 24.716470 | 389.913900 | 0.153504 | 3.60212 | 2.47 |
| 25.215700 | 340.009100 | 0.153504 | 3.53192 | 2.16 |
| 25.528630 | 100.247600 | 0.102336 | 3.48933 | 0.64 |
| 26.555510 | 851.397900 | 0.127920 | 3.35669 | 5.40 |
| 27.495570 | 346.388400 | 0.102336 | 3.24402 | 2.20 |
| 28.492430 | 151.165100 | 0.153504 | 3.13275 | 0.96 |
| 29.042300 | 127.460400 | 0.127920 | 3.07468 | 0.81 |
| 29.493730 | 31.629500 | 0.102336 | 3.02864 | 0.20 |
| 30.031030 | 264.662000 | 0.127920 | 2.97567 | 1.68 |
| 30.281130 | 477.097800 | 0.153504 | 2.95166 | 3.02 |
| 30.807330 | 72.322240 | 0.153504 | 2.90243 | 0.46 |
| 31.997810 | 60.348350 | 0.153504 | 2.79711 | 0.38 |
| 35.225640 | 18.970190 | 0.102336 | 2.54785 | 0.12 |
| 36.879590 | 55.833400 | 0.307008 | 2.43730 | 0.35 |
| 38.025720 | 55.632700 | 0.204672 | 2.36643 | 0.35 |
| 39.081850 | 40.755120 | 0.102336 | 2.30488 | 0.26 |

Form C:

| | | | | |
|---|---|---|---|---|
| 3.194969 | 35.615380 | 0.204672 | 27.65420 | 0.70 |
| 3.812396 | 716.015100 | 0.230256 | 23.17681 | 14.14 |
| 5.856102 | 1436.277000 | 0.179088 | 15.09218 | 28.37 |
| 6.456882 | 5063.125000 | 0.230256 | 13.68922 | 100.00 |
| 7.459860 | 1033.798000 | 0.204672 | 11.85080 | 20.42 |
| 9.850815 | 810.558400 | 0.179088 | 8.97913 | 16.01 |
| 11.187440 | 62.419810 | 0.153504 | 7.90917 | 1.23 |
| 12.939770 | 245.169100 | 0.102336 | 6.84177 | 4.84 |
| 13.494440 | 75.203870 | 0.102336 | 6.56177 | 1.49 |
| 14.497570 | 232.105300 | 0.102336 | 6.10992 | 4.58 |
| 14.937460 | 329.660100 | 0.204672 | 5.93096 | 6.51 |
| 15.433590 | 461.171900 | 0.179088 | 5.74141 | 9.11 |
| 15.879330 | 2886.998000 | 0.153504 | 5.58123 | 57.02 |
| 17.171920 | 689.987900 | 0.204672 | 5.16392 | 13.63 |
| 17.945050 | 496.922600 | 0.204672 | 4.94315 | 9.81 |
| 19.074500 | 798.566900 | 0.127920 | 4.65292 | 15.77 |
| 19.441190 | 2391.910000 | 0.204672 | 4.56598 | 47.24 |
| 20.837700 | 578.154500 | 0.204672 | 4.26303 | 11.42 |
| 21.534950 | 368.731800 | 0.102336 | 4.12655 | 7.28 |
| 22.148020 | 940.696900 | 0.204672 | 4.01369 | 18.58 |
| 22.774290 | 749.816500 | 0.204672 | 3.90472 | 14.81 |
| 23.338800 | 350.197500 | 0.127920 | 3.81153 | 6.92 |
| 24.235250 | 408.793000 | 0.179088 | 3.67254 | 8.07 |
| 25.108530 | 363.560800 | 0.179088 | 3.54675 | 7.18 |
| 26.236280 | 811.929100 | 0.179088 | 3.39680 | 16.04 |
| 27.380220 | 312.011300 | 0.204672 | 3.25743 | 6.16 |
| 28.411830 | 311.342200 | 0.153504 | 3.14146 | 6.15 |
| 29.612520 | 95.846000 | 0.409344 | 3.01676 | 1.89 |
| 30.077750 | 88.930350 | 0.179088 | 2.97115 | 1.76 |
| 30.567640 | 270.615100 | 0.153504 | 2.92464 | 5.34 |
| 31.335150 | 50.665160 | 0.153504 | 2.85473 | 1.00 |
| 31.715630 | 111.390500 | 0.204672 | 2.82135 | 2.20 |
| 32.610040 | 93.354220 | 0.255840 | 2.74598 | 1.84 |
| 33.141310 | 70.288350 | 0.204672 | 2.70317 | 1.39 |
| 33.990470 | 56.458010 | 0.307008 | 2.63756 | 1.12 |
| 35.198830 | 96.069080 | 0.204672 | 2.54973 | 1.90 |
| 35.775650 | 67.623210 | 0.255840 | 2.50993 | 1.34 |
| 37.471480 | 26.640010 | 0.614016 | 2.40015 | 0.53 |
| 38.903870 | 83.803490 | 0.204672 | 2.31502 | 1.66 |
| 39.217410 | 57.555520 | 0.153504 | 2.29723 | 1.14 |

Form D:

| | | | | |
|---|---|---|---|---|
| 3.153332 | 169.307800 | 0.204672 | 28.01926 | 0.50 |
| 4.431448 | 2658.429000 | 0.127920 | 19.94041 | 7.86 |
| 5.941830 | 33835.610000 | 0.127920 | 14.87462 | 100.00 |
| 8.426903 | 1065.092000 | 0.102336 | 10.49289 | 3.15 |
| 8.816577 | 3616.264000 | 0.102336 | 10.02998 | 10.69 |
| 9.383790 | 375.809400 | 0.127920 | 9.42494 | 1.11 |
| 10.439640 | 807.970000 | 0.102336 | 8.47397 | 2.39 |
| 11.271880 | 7283.616000 | 0.127920 | 7.85011 | 21.53 |
| 11.857220 | 5193.201000 | 0.102336 | 7.46387 | 15.35 |
| 12.078650 | 1768.847000 | 0.076752 | 7.32753 | 5.23 |
| 12.338430 | 2233.939000 | 0.127920 | 7.17383 | 6.60 |
| 12.987940 | 115.890800 | 0.127920 | 6.81650 | 0.34 |
| 13.219940 | 357.182200 | 0.102336 | 6.69739 | 1.06 |
| 13.618540 | 13.893700 | 0.409344 | 6.50225 | 0.04 |
| 13.900400 | 467.011200 | 0.102336 | 6.37103 | 1.38 |
| 14.725480 | 569.724400 | 0.153504 | 6.01586 | 1.68 |
| 15.152470 | 487.087300 | 0.102336 | 5.84729 | 1.44 |
| 15.353710 | 286.600400 | 0.076752 | 5.77110 | 0.85 |
| 15.777290 | 323.654600 | 0.102336 | 5.61710 | 0.96 |
| 16.110340 | 1169.031000 | 0.102336 | 5.50172 | 3.46 |
| 16.886170 | 1272.457000 | 0.102336 | 5.25065 | 3.76 |
| 17.196420 | 328.858300 | 0.102336 | 5.15662 | 0.97 |
| 17.812720 | 513.725300 | 0.153504 | 4.97957 | 1.52 |
| 18.528180 | 1350.473000 | 0.102336 | 4.78887 | 3.99 |
| 18.743030 | 261.212500 | 0.076752 | 4.73445 | 0.77 |
| 19.213330 | 756.393700 | 0.102336 | 4.61961 | 2.24 |
| 19.414990 | 2298.519000 | 0.102336 | 4.57208 | 6.79 |
| 19.657140 | 2311.595000 | 0.127920 | 4.51630 | 6.83 |
| 20.031570 | 756.401100 | 0.102336 | 4.43272 | 2.24 |
| 20.246710 | 175.839200 | 0.076752 | 4.38611 | 0.52 |
| 20.747560 | 4468.067000 | 0.102336 | 4.28134 | 13.21 |
| 20.976770 | 1522.862000 | 0.102336 | 4.23508 | 4.50 |
| 21.834720 | 531.257800 | 0.153504 | 4.07057 | 1.57 |
| 22.393390 | 266.612300 | 0.102336 | 3.97026 | 0.79 |
| 22.792050 | 2442.323000 | 0.127920 | 3.90171 | 7.22 |
| 23.315150 | 266.353400 | 0.076752 | 3.81535 | 0.79 |
| 23.609490 | 121.638500 | 0.076752 | 3.76844 | 0.36 |
| 24.051330 | 255.356300 | 0.102336 | 3.70020 | 0.75 |
| 24.739830 | 2664.119000 | 0.179088 | 3.59877 | 7.87 |
| 25.164060 | 436.465200 | 0.127920 | 3.53905 | 1.29 |
| 25.661990 | 87.449390 | 0.153504 | 3.47150 | 0.26 |
| 25.967390 | 142.152200 | 0.102336 | 3.43136 | 0.42 |
| 26.553990 | 365.973800 | 0.127920 | 3.35688 | 1.08 |
| 26.913950 | 1881.958000 | 0.153504 | 3.31279 | 5.56 |
| 27.307290 | 314.477400 | 0.127920 | 3.26596 | 0.93 |
| 28.303020 | 72.745290 | 0.076752 | 3.15329 | 0.21 |
| 28.565290 | 561.713000 | 0.127920 | 3.12493 | 1.66 |
| 29.083400 | 35.792210 | 0.127920 | 3.07043 | 0.11 |
| 29.601760 | 255.646800 | 0.179088 | 3.01783 | 0.76 |
| 29.877580 | 151.904800 | 0.127920 | 2.99060 | 0.45 |

Form E:

| | | | | |
|---|---|---|---|---|
| 3.223808 | 254.574600 | 0.179088 | 27.40688 | 2.75 |
| 4.545509 | 66.329070 | 0.307008 | 19.44030 | 0.72 |
| 6.624407 | 9269.059000 | 0.127920 | 13.34340 | 100.00 |
| 8.007524 | 4108.179000 | 0.153504 | 11.04147 | 44.32 |
| 8.949897 | 4742.364000 | 0.102336 | 9.88087 | 51.16 |
| 9.678118 | 69.981640 | 0.102336 | 9.13897 | 0.76 |
| 10.222920 | 298.882400 | 0.127920 | 8.65312 | 3.22 |
| 11.003670 | 14.221230 | 0.409344 | 8.04084 | 0.15 |
| 11.635970 | 43.491710 | 0.102336 | 7.60528 | 0.47 |
| 11.970480 | 44.075680 | 0.076752 | 7.39350 | 0.48 |
| 12.365720 | 106.049700 | 0.076752 | 7.15806 | 1.14 |
| 12.819600 | 1294.077000 | 0.102336 | 6.90563 | 13.96 |
| 13.205330 | 103.229400 | 0.204672 | 6.70477 | 1.11 |
| 13.857730 | 368.166600 | 0.153504 | 6.39056 | 3.97 |
| 15.944810 | 423.062300 | 0.153504 | 5.55846 | 4.56 |
| 16.288310 | 1540.268000 | 0.127920 | 5.44201 | 16.62 |
| 17.265660 | 31.821440 | 0.076752 | 5.13609 | 0.34 |
| 17.939710 | 2597.920000 | 0.102336 | 4.94460 | 28.03 |
| 18.832640 | 1204.338000 | 0.179088 | 4.71213 | 12.99 |
| 19.469820 | 105.158300 | 0.307008 | 4.55933 | 1.13 |
| 20.616500 | 993.015900 | 0.204672 | 4.30826 | 10.71 |
| 22.296370 | 2354.107000 | 0.153504 | 3.98732 | 25.40 |
| 23.448510 | 1027.299000 | 0.153504 | 3.79395 | 11.08 |
| 24.312890 | 1658.200000 | 0.127920 | 3.66099 | 17.89 |
| 25.050100 | 92.009680 | 0.153504 | 3.55489 | 0.99 |
| 25.649440 | 343.220300 | 0.127920 | 3.47317 | 3.70 |
| 26.110090 | 1003.525000 | 0.230256 | 3.41293 | 10.83 |
| 26.588660 | 86.151680 | 0.153504 | 3.35258 | 0.93 |
| 26.968050 | 120.633700 | 0.204672 | 3.30627 | 1.30 |
| 27.560610 | 105.635400 | 0.255840 | 3.23652 | 1.14 |
| 28.575280 | 141.872600 | 0.102336 | 3.12386 | 1.53 |
| 29.730410 | 64.201560 | 0.255840 | 3.00506 | 0.69 |
| 30.309700 | 54.688740 | 0.230256 | 2.94894 | 0.59 |
| 30.942090 | 18.423020 | 0.307008 | 2.89010 | 0.20 |
| 31.864190 | 66.645730 | 0.153504 | 2.80854 | 0.72 |
| 32.386040 | 193.558800 | 0.153504 | 2.76446 | 2.09 |
| 33.072680 | 71.152350 | 0.153504 | 2.70862 | 0.77 |
| 33.849360 | 65.444690 | 0.127920 | 2.64823 | 0.71 |
| 34.437140 | 88.265690 | 0.204672 | 2.60436 | 0.95 |
| 36.312720 | 22.743430 | 0.153504 | 2.47403 | 0.25 |
| 37.628940 | 8.127533 | 0.409344 | 2.39047 | 0.09 |
| 38.771420 | 25.264060 | 0.307008 | 2.32262 | 0.27 |
| 39.366760 | 34.209900 | 0.204672 | 2.28885 | 0.37 |

Form F:

| | | | | |
|---|---|---|---|---|
| 3.178694 | 45.813100 | 0.076752 | 27.79576 | 4.64 |
| 3.325501 | 29.859660 | 0.076752 | 26.56901 | 3.02 |
| 4.168773 | 63.817540 | 0.076752 | 21.19625 | 6.46 |
| 4.434412 | 45.639750 | 0.102336 | 19.92709 | 4.62 |
| 4.965300 | 46.610870 | 0.076752 | 17.79762 | 4.72 |
| 5.658827 | 174.513200 | 0.358176 | 15.61786 | 17.67 |
| 6.419788 | 45.377340 | 0.063960 | 13.76823 | 4.60 |
| 7.512460 | 987.422200 | 0.230256 | 11.76794 | 100.00 |
| 7.891961 | 525.677400 | 0.127920 | 11.20289 | 53.24 |
| 9.262142 | 177.934000 | 0.102336 | 9.54845 | 18.02 |
| 9.638004 | 105.897100 | 0.051168 | 9.17691 | 10.72 |
| 9.789849 | 41.722350 | 0.102336 | 9.03491 | 4.23 |
| 11.222670 | 223.954900 | 0.153504 | 7.88442 | 22.68 |
| 11.735000 | 202.417800 | 0.255840 | 7.54133 | 20.50 |
| 12.110230 | 39.485890 | 0.038376 | 7.30850 | 4.00 |
| 12.220780 | 55.438760 | 0.051168 | 7.24263 | 5.61 |
| 12.529300 | 118.171200 | 0.102336 | 7.06497 | 11.97 |
| 13.365880 | 44.173050 | 0.051168 | 6.62459 | 4.47 |
| 13.505930 | 48.023010 | 0.102336 | 6.55621 | 4.86 |
| 14.321600 | 58.829660 | 0.102336 | 6.18459 | 5.96 |
| 15.690800 | 66.869320 | 0.038376 | 5.64787 | 6.77 |
| 16.914250 | 63.279140 | 0.063960 | 5.24200 | 6.41 |
| 17.199370 | 38.307210 | 0.038376 | 5.15574 | 3.88 |
| 17.625100 | 249.335100 | 0.127920 | 5.03215 | 25.25 |
| 18.952340 | 404.319200 | 0.358176 | 4.68264 | 40.95 |
| 20.252680 | 312.856600 | 0.614016 | 4.38483 | 31.68 |
| 21.896730 | 3.386084 | 0.051168 | 4.05918 | 0.34 |
| 22.367480 | 530.229800 | 0.127920 | 3.97480 | 53.70 |
| 23.129520 | 179.857100 | 0.255840 | 3.84554 | 18.21 |
| 23.804160 | 44.651970 | 0.102336 | 3.73806 | 4.52 |
| 25.040020 | 27.817500 | 0.153504 | 3.55630 | 2.82 |
| 25.723530 | 39.814260 | 0.127920 | 3.46334 | 4.03 |
| 27.160890 | 28.098980 | 0.076752 | 3.28323 | 2.85 |
| 28.114280 | 23.322130 | 0.153504 | 3.17402 | 2.36 |
| 29.388560 | 30.545480 | 0.051168 | 3.03924 | 3.09 |
| 30.289680 | 33.744010 | 0.076752 | 2.95084 | 3.42 |
| 31.931690 | 26.935100 | 0.063960 | 2.80275 | 2.73 |
| 33.508710 | 9.988585 | 0.051168 | 2.67437 | 1.01 |
| 37.127620 | 36.166680 | 0.046800 | 2.41958 | 3.66 |
| 38.118730 | 35.280220 | 0.051168 | 2.36087 | 3.57 |

Form G:

| | | | | |
|---|---|---|---|---|
| 3.057389 | 29.327710 | 0.102336 | 28.89831 | 1.12 |
| 3.320305 | 12.952490 | 0.102336 | 26.61058 | 0.50 |
| 4.125527 | 22.215040 | 0.102336 | 21.41835 | 0.85 |
| 4.673285 | 19.682430 | 0.127920 | 18.90905 | 0.75 |
| 6.283435 | 600.213900 | 0.051168 | 14.06669 | 22.97 |
| 7.780389 | 52.321620 | 0.204672 | 11.36329 | 2.00 |
| 8.357451 | 104.135600 | 0.153504 | 10.57993 | 3.99 |
| 9.034883 | 15.194670 | 0.153504 | 9.78812 | 0.58 |
| 10.884600 | 128.167800 | 0.179088 | 8.12854 | 4.91 |
| 12.336040 | 30.377210 | 0.076752 | 7.17522 | 1.16 |
| 12.563210 | 93.844090 | 0.038376 | 7.04598 | 3.59 |
| 13.789700 | 10.633010 | 0.307008 | 6.42193 | 0.41 |
| 14.497210 | 7.484401 | 0.409344 | 6.11007 | 0.29 |
| 15.563210 | 15.552420 | 0.511680 | 5.69388 | 0.60 |
| 16.632960 | 70.214870 | 0.102336 | 5.33001 | 2.69 |
| 17.603200 | 39.619290 | 0.307008 | 5.03836 | 1.52 |
| 18.389760 | 53.215650 | 0.127920 | 4.82460 | 2.04 |
| 18.855700 | 2612.816000 | 0.051168 | 4.70642 | 100.00 |
| 19.746090 | 101.330000 | 0.204672 | 4.49616 | 3.88 |
| 20.512260 | 86.700270 | 0.102336 | 4.32992 | 3.32 |
| 21.214370 | 64.815360 | 0.127920 | 4.18818 | 2.48 |
| 21.952470 | 137.321300 | 0.102336 | 4.04900 | 5.26 |
| 22.737490 | 78.777340 | 0.255840 | 3.91095 | 3.02 |
| 23.399160 | 29.378910 | 0.127920 | 3.80184 | 1.12 |
| 23.925380 | 24.954240 | 0.153504 | 3.71940 | 0.96 |
| 24.494490 | 40.232560 | 0.307008 | 3.63426 | 1.54 |
| 25.224590 | 689.014000 | 0.038376 | 3.53070 | 26.37 |
| 25.864670 | 23.144240 | 0.153504 | 3.44476 | 0.89 |
| 26.462280 | 13.818680 | 0.307008 | 3.36830 | 0.53 |
| 28.208050 | 21.898060 | 0.204672 | 3.16369 | 0.84 |
| 29.011920 | 37.525020 | 0.127920 | 3.07783 | 1.44 |
| 29.599420 | 12.601030 | 0.153504 | 3.01806 | 0.48 |
| 30.217880 | 16.616070 | 0.307008 | 2.95769 | 0.64 |
| 31.673660 | 249.517600 | 0.038376 | 2.82499 | 9.55 |
| 32.620170 | 6.461364 | 0.409344 | 2.74515 | 0.25 |
| 38.226000 | 350.608600 | 0.046800 | 2.35254 | 13.42 |

Form H:

| | | | | |
|---|---|---|---|---|
| 3.099136 | 19.949290 | 0.204672 | 28.50912 | 1.47 |
| 3.646530 | 16.577460 | 0.076752 | 24.23065 | 1.23 |
| 4.363048 | 73.752250 | 0.127920 | 20.25286 | 5.45 |
| 5.727725 | 1352.822000 | 0.076752 | 15.43015 | 100.00 |
| 6.008395 | 81.391620 | 0.076752 | 14.70998 | 6.02 |
| 6.655096 | 8.122511 | 0.307008 | 13.28194 | 0.60 |
| 8.232493 | 13.942910 | 0.153504 | 10.74024 | 1.03 |
| 8.725933 | 76.479350 | 0.127920 | 10.13397 | 5.65 |
| 10.322960 | 50.104050 | 0.127920 | 8.56948 | 3.70 |
| 10.898240 | 446.715400 | 0.076752 | 8.11839 | 33.02 |
| 11.446050 | 388.088200 | 0.076752 | 7.73105 | 28.69 |
| 12.022070 | 104.122300 | 0.051168 | 7.36189 | 7.70 |
| 12.817090 | 20.923400 | 0.076752 | 6.90698 | 1.55 |
| 13.508090 | 46.248000 | 0.076752 | 6.55517 | 3.42 |
| 14.360230 | 20.274310 | 0.204672 | 6.16804 | 1.50 |
| 15.895580 | 97.727670 | 0.089544 | 5.57556 | 7.22 |
| 16.426840 | 95.441180 | 0.127920 | 5.39642 | 7.05 |
| 16.675810 | 316.096300 | 0.089544 | 5.31641 | 23.37 |
| 17.245350 | 52.702640 | 0.127920 | 5.14209 | 3.90 |
| 18.071070 | 18.371720 | 0.204672 | 4.90896 | 1.36 |
| 18.430560 | 32.563690 | 0.153504 | 4.81401 | 2.41 |
| 18.857260 | 237.783200 | 0.076752 | 4.70603 | 17.58 |
| 19.119920 | 321.130500 | 0.089544 | 4.64197 | 23.74 |
| 19.562450 | 23.032940 | 0.153504 | 4.53795 | 1.70 |
| 19.864540 | 63.697700 | 0.102336 | 4.46962 | 4.71 |
| 20.352290 | 234.578600 | 0.076752 | 4.36359 | 17.34 |
| 20.837090 | 74.304350 | 0.102336 | 4.26315 | 5.49 |
| 21.254610 | 54.243780 | 0.127920 | 4.18034 | 4.01 |
| 22.027940 | 143.167800 | 0.076752 | 4.03530 | 10.58 |
| 22.619560 | 98.559720 | 0.051168 | 3.93107 | 7.29 |
| 22.892640 | 60.932400 | 0.102336 | 3.88480 | 4.50 |
| 23.226790 | 60.662960 | 0.127920 | 3.82966 | 4.48 |
| 23.955300 | 157.354500 | 0.127920 | 3.71482 | 11.63 |
| 24.175430 | 106.334500 | 0.127920 | 3.68149 | 7.86 |
| 24.811950 | 23.558100 | 0.102336 | 3.58847 | 1.74 |
| 25.974150 | 29.833640 | 0.127920 | 3.43048 | 2.21 |
| 26.357980 | 154.997100 | 0.102336 | 3.38139 | 11.46 |
| 27.694480 | 7.210134 | 0.307008 | 3.22118 | 0.53 |
| 28.735050 | 34.483060 | 0.102336 | 3.10685 | 2.55 |
| 28.977000 | 39.221290 | 0.153504 | 3.08146 | 2.90 |
| 29.376030 | 34.110350 | 0.102336 | 3.04050 | 2.52 |
| 30.272620 | 35.313870 | 0.153504 | 2.95247 | 2.61 |
| 33.063500 | 21.608550 | 0.127920 | 2.70935 | 1.60 |
| 33.698660 | 37.710240 | 0.153504 | 2.65972 | 2.79 |
| 38.960580 | 10.923690 | 0.409344 | 2.31178 | 0.81 |

Form I:

| | | | | |
|---|---|---|---|---|
| 3.781225 | 26.785220 | 0.051168 | 23.36780 | 2.08 |
| 4.760598 | 11.119640 | 0.153504 | 18.56244 | 0.86 |
| 5.401361 | 51.423460 | 0.153504 | 16.36173 | 4.00 |
| 6.277009 | 1286.643000 | 0.063960 | 14.08108 | 100.00 |
| 7.782325 | 365.825000 | 0.063960 | 11.36046 | 28.43 |
| 8.430950 | 885.338700 | 0.063960 | 10.48786 | 68.81 |
| 8.998234 | 108.646500 | 0.051168 | 9.82790 | 8.44 |
| 9.893166 | 12.216860 | 0.102336 | 8.94079 | 0.95 |
| 10.390060 | 141.699500 | 0.102336 | 8.51429 | 11.01 |
| 11.774070 | 289.927200 | 0.076752 | 7.51639 | 22.53 |
| 12.477330 | 14.584140 | 0.076752 | 7.09428 | 1.13 |
| 13.787140 | 23.490670 | 0.153504 | 6.42311 | 1.83 |
| 14.235030 | 89.408000 | 0.063960 | 6.22201 | 6.95 |
| 15.254360 | 77.687270 | 0.153504 | 5.80846 | 6.04 |
| 15.558040 | 121.010100 | 0.076752 | 5.69576 | 9.41 |
| 16.472210 | 62.644770 | 0.076752 | 5.38166 | 4.87 |
| 17.179380 | 560.647900 | 0.063960 | 5.16169 | 43.57 |
| 17.961650 | 188.601000 | 0.076752 | 4.93861 | 14.66 |
| 18.774440 | 792.503100 | 0.063960 | 4.72661 | 61.59 |
| 19.403260 | 199.829000 | 0.102336 | 4.57482 | 15.53 |
| 20.842530 | 68.443550 | 0.102336 | 4.26205 | 5.32 |
| 21.021690 | 57.874100 | 0.127920 | 4.22613 | 4.50 |
| 21.514130 | 345.837500 | 0.076752 | 4.13050 | 26.88 |
| 22.996520 | 88.302530 | 0.102336 | 3.86748 | 6.86 |
| 25.552010 | 135.955100 | 0.102336 | 3.48619 | 10.57 |
| 26.471860 | 51.841460 | 0.204672 | 3.36711 | 4.03 |
| 27.026890 | 14.750680 | 0.204672 | 3.29921 | 1.15 |
| 29.237340 | 22.381110 | 0.179088 | 3.05461 | 1.74 |
| 30.129540 | 24.333990 | 0.307008 | 2.96616 | 1.89 |
| 36.834370 | 6.560548 | 0.614016 | 2.44019 | 0.51 |

Form J:

| | | | | |
|---|---|---|---|---|
| 3.060036 | 20.107240 | 0.076752 | 28.87331 | 1.24 |
| 3.546805 | 12.909750 | 0.063960 | 24.91171 | 0.80 |
| 5.589293 | 1623.410000 | 0.038376 | 15.81200 | 100.00 |
| 7.172551 | 30.510520 | 0.076752 | 12.32485 | 1.88 |
| 9.346706 | 151.934600 | 0.127920 | 9.46225 | 9.36 |
| 10.228600 | 12.634670 | 0.255840 | 8.64833 | 0.78 |
| 11.157600 | 30.001350 | 0.153504 | 7.93026 | 1.85 |
| 12.433120 | 28.395290 | 0.179088 | 7.11941 | 1.75 |
| 14.807790 | 9.132507 | 0.818688 | 5.98261 | 0.56 |
| 16.174290 | 33.692260 | 0.127920 | 5.48011 | 2.08 |
| 16.761260 | 488.234000 | 0.076752 | 5.28950 | 30.07 |
| 18.104100 | 39.459320 | 0.153504 | 4.90007 | 2.43 |
| 18.779020 | 58.463190 | 0.179088 | 4.72546 | 3.60 |
| 19.593020 | 17.123870 | 0.307008 | 4.53094 | 1.05 |
| 20.545190 | 8.742917 | 0.204672 | 4.32305 | 0.54 |
| 21.112540 | 49.452260 | 0.153504 | 4.20815 | 3.05 |
| 21.638240 | 70.180120 | 0.255840 | 4.10708 | 4.32 |
| 22.429600 | 95.639860 | 0.153504 | 3.96394 | 5.89 |
| 24.051750 | 48.808610 | 0.204672 | 3.70014 | 3.01 |
| 24.619970 | 39.523670 | 0.153504 | 3.61602 | 2.43 |
| 25.750420 | 74.033410 | 0.153504 | 3.45978 | 4.56 |
| 26.302590 | 24.439390 | 0.051168 | 3.38839 | 1.51 |
| 26.634740 | 59.148650 | 0.307008 | 3.34688 | 3.64 |
| 28.131650 | 13.329710 | 0.307008 | 3.17210 | 0.82 |
| 32.319550 | 16.882490 | 0.204672 | 2.77000 | 1.04 |
| 33.918590 | 12.353660 | 0.153504 | 2.64298 | 0.76 |
| 34.822170 | 11.704460 | 0.076752 | 2.57644 | 0.72 |
| 35.180100 | 5.877381 | 0.409344 | 2.55105 | 0.36 |

Form K:

| | | | | |
|---|---|---|---|---|
| 3.125749 | 8.088668 | 0.204672 | 28.26645 | 0.20 |
| 3.690096 | 34.221650 | 0.038376 | 23.94468 | 0.86 |
| 6.655328 | 4000.436000 | 0.102336 | 13.28147 | 100.00 |
| 6.918468 | 2274.822000 | 0.153504 | 12.77690 | 56.86 |
| 9.126177 | 78.247190 | 0.127920 | 9.69040 | 1.96 |
| 9.939386 | 170.294900 | 0.102336 | 8.89932 | 4.26 |
| 13.794490 | 148.119900 | 0.102336 | 6.41971 | 3.70 |
| 14.462850 | 9.473206 | 0.204672 | 6.12451 | 0.24 |
| 15.134850 | 287.340900 | 0.102336 | 5.85405 | 7.18 |
| 15.552060 | 388.102900 | 0.179088 | 5.69794 | 9.70 |
| 17.277180 | 38.765110 | 0.102336 | 5.13269 | 0.97 |
| 17.892630 | 169.488900 | 0.179088 | 4.95751 | 4.24 |
| 18.715640 | 17.159640 | 0.204672 | 4.74132 | 0.43 |
| 20.016740 | 395.259800 | 0.127920 | 4.43598 | 9.88 |
| 20.708050 | 26.209950 | 0.204672 | 4.28942 | 0.66 |
| 21.277530 | 47.009900 | 0.204672 | 4.17589 | 1.18 |
| 22.016590 | 40.886010 | 0.255840 | 4.03735 | 1.02 |
| 22.500280 | 11.137440 | 0.204672 | 3.95164 | 0.28 |
| 23.319700 | 15.363510 | 0.076752 | 3.81461 | 0.38 |
| 23.819170 | 14.342480 | 0.204672 | 3.73574 | 0.36 |
| 24.886350 | 38.284170 | 0.179088 | 3.57791 | 0.96 |
| 25.802420 | 18.001050 | 0.307008 | 3.45293 | 0.45 |
| 26.744750 | 587.459500 | 0.115128 | 3.33337 | 14.68 |
| 27.751720 | 378.932300 | 0.127920 | 3.21466 | 9.47 |
| 28.685780 | 14.706780 | 0.204672 | 3.11208 | 0.37 |
| 29.261460 | 10.540700 | 0.255840 | 3.05215 | 0.26 |
| 30.570120 | 118.664600 | 0.179088 | 2.92441 | 2.97 |
| 31.603030 | 55.510100 | 0.307008 | 2.83115 | 1.39 |
| 33.571690 | 29.434850 | 0.255840 | 2.66949 | 0.74 |
| 35.121890 | 19.828520 | 0.511680 | 2.55514 | 0.50 |
| 37.437180 | 28.227860 | 0.102336 | 2.40227 | 0.71 |
| 38.211490 | 10.843290 | 0.153504 | 2.35535 | 0.27 |
| 38.760920 | 23.425870 | 0.255840 | 2.32322 | 0.59 |

Form L:

| | | | | |
|---|---|---|---|---|
| 3.114982 | 14.554110 | 0.153504 | 28.36413 | 0.35 |
| 3.758476 | 14.447550 | 0.409344 | 23.50919 | 0.35 |
| 4.169389 | 5.237628 | 0.038376 | 21.19312 | 0.13 |
| 4.260997 | 16.627330 | 0.051168 | 20.73769 | 0.40 |
| 5.242640 | 550.946700 | 0.127920 | 16.85672 | 13.32 |
| 6.074583 | 4135.564000 | 0.191880 | 14.54985 | 100.00 |
| 6.651309 | 1421.657000 | 0.230256 | 13.28949 | 34.38 |
| 8.033223 | 54.731720 | 0.038376 | 11.00620 | 1.32 |
| 8.987103 | 33.490450 | 0.153504 | 9.84005 | 0.81 |
| 11.092880 | 10.653230 | 0.255840 | 7.97638 | 0.26 |
| 12.662410 | 6.889622 | 0.614016 | 6.99100 | 0.17 |
| 14.958270 | 14.251120 | 0.409344 | 5.92276 | 0.34 |
| 15.403210 | 14.595950 | 0.076752 | 5.75266 | 0.35 |
| 16.002700 | 31.538320 | 0.127920 | 5.53848 | 0.76 |
| 17.989470 | 35.317420 | 0.153504 | 4.93104 | 0.85 |
| 18.896130 | 12.980740 | 0.204672 | 4.69644 | 0.31 |
| 20.636900 | 528.174400 | 0.153504 | 4.30405 | 12.77 |
| 22.379920 | 35.571950 | 0.204672 | 3.97262 | 0.86 |
| 23.203550 | 23.980560 | 0.255840 | 3.83344 | 0.58 |
| 25.413920 | 9.127674 | 0.307008 | 3.50482 | 0.22 |
| 26.798530 | 21.352080 | 0.409344 | 3.32680 | 0.52 |
| 33.827310 | 8.121967 | 0.153504 | 2.64990 | 0.20 |

Form M:

| | | | | |
|---|---|---|---|---|
| 4.728935 | 13.776310 | 0.818688 | 18.68665 | 4.03 |
| 6.588024 | 341.719900 | 0.051168 | 13.41701 | 100.00 |
| 8.261230 | 75.353040 | 0.076752 | 10.70294 | 22.05 |
| 12.250490 | 9.965623 | 0.409344 | 7.22513 | 2.92 |
| 13.289440 | 6.679496 | 0.307008 | 6.66252 | 1.95 |
| 14.378050 | 17.072970 | 0.255840 | 6.16044 | 5.00 |
| 16.397870 | 6.512505 | 0.409344 | 5.40589 | 1.91 |
| 17.840790 | 24.415730 | 0.153504 | 4.97180 | 7.14 |
| 19.791230 | 48.229930 | 0.076752 | 4.48601 | 14.11 |
| 20.690970 | 7.122862 | 0.307008 | 4.29292 | 2.08 |
| 22.710500 | 36.524030 | 0.204672 | 3.91554 | 10.69 |
| 24.566370 | 12.601660 | 0.614016 | 3.62378 | 3.69 |
| 26.487140 | 24.771800 | 0.153504 | 3.36520 | 7.25 |

Form N:

| | | | | |
|---|---|---|---|---|
| 3.113648 | 376.837200 | 0.102336 | 28.37628 | 16.12 |
| 3.318589 | 594.056600 | 0.127920 | 26.62434 | 25.41 |
| 4.161664 | 912.430700 | 0.204672 | 21.23245 | 39.03 |
| 4.630490 | 459.210400 | 0.460512 | 19.08371 | 19.64 |
| 6.117166 | 2337.646000 | 0.460512 | 14.44866 | 100.00 |
| 9.118856 | 132.685900 | 0.614016 | 9.69817 | 5.68 |
| 9.931746 | 174.102600 | 0.511680 | 8.90615 | 7.45 |
| 11.301470 | 32.516500 | 0.127920 | 7.82962 | 1.39 |
| 12.795170 | 94.187770 | 0.716352 | 6.91876 | 4.03 |
| 14.601680 | 66.507620 | 0.204672 | 6.06659 | 2.85 |
| 16.650740 | 576.411700 | 0.204672 | 5.32436 | 24.66 |
| 18.046860 | 122.791300 | 0.614016 | 4.91549 | 5.25 |
| 19.290020 | 675.995700 | 0.409344 | 4.60142 | 28.92 |
| 21.812730 | 236.414100 | 0.818688 | 4.07462 | 10.11 |
| 22.454700 | 190.607200 | 0.614016 | 3.95956 | 8.15 |
| 24.552440 | 82.365620 | 0.409344 | 3.62581 | 3.52 |
| 25.178850 | 69.694710 | 0.076752 | 3.53701 | 2.98 |
| 26.198530 | 180.769800 | 0.614016 | 3.40161 | 7.73 |
| 26.644090 | 97.681620 | 0.076752 | 3.34573 | 4.18 |
| 27.768210 | 54.726810 | 0.076752 | 3.21279 | 2.34 |
| 29.782660 | 103.983000 | 0.409344 | 2.99991 | 4.45 |
| 31.465190 | 40.332150 | 0.076752 | 2.84323 | 1.73 |
| 32.055110 | 60.119980 | 0.102336 | 2.79224 | 2.57 |
| 32.371410 | 56.691350 | 0.409344 | 2.76568 | 2.43 |
| 33.063660 | 46.334440 | 0.127920 | 2.70934 | 1.98 |
| 34.070330 | 25.037890 | 0.153504 | 2.63156 | 1.07 |
| 34.636520 | 16.466960 | 0.255840 | 2.58983 | 0.70 |
| 36.130640 | 19.433070 | 0.614016 | 2.48608 | 0.83 |
| 37.015120 | 29.113220 | 0.076752 | 2.42869 | 1.25 |
| 37.664140 | 9.674776 | 0.614016 | 2.38831 | 0.41 |
| 38.024600 | 22.310820 | 0.102336 | 2.36650 | 0.95 |

Form O:

| | | | | |
|---|---|---|---|---|
| 3.118466 | 24.847240 | 0.204672 | 28.33245 | 0.12 |
| 5.589300 | 21028.510000 | 0.063960 | 15.81198 | 100.00 |
| 6.629287 | 1108.200000 | 0.140712 | 13.33359 | 5.27 |
| 11.158960 | 255.084800 | 0.076752 | 7.92929 | 1.21 |
| 11.890180 | 74.750580 | 0.102336 | 7.44325 | 0.36 |
| 12.501050 | 22.268180 | 0.153504 | 7.08088 | 0.11 |
| 12.790830 | 30.640750 | 0.127920 | 6.92110 | 0.15 |
| 13.679850 | 31.283680 | 0.102336 | 6.47325 | 0.15 |
| 13.931390 | 18.834180 | 0.051168 | 6.35693 | 0.09 |
| 14.526300 | 31.686580 | 0.127920 | 6.09790 | 0.15 |
| 15.138950 | 35.324620 | 0.063960 | 5.85248 | 0.17 |
| 16.199310 | 73.933690 | 0.127920 | 5.47170 | 0.35 |
| 16.760720 | 17539.250000 | 0.076752 | 5.28967 | 83.41 |
| 18.091150 | 58.280900 | 0.127920 | 4.90355 | 0.28 |
| 18.491030 | 20.503230 | 0.038376 | 4.79840 | 0.10 |
| 18.779640 | 25.379980 | 0.153504 | 4.72531 | 0.12 |
| 19.183380 | 18.742030 | 0.127920 | 4.62676 | 0.09 |
| 19.476950 | 12.469360 | 0.127920 | 4.55768 | 0.06 |
| 20.014390 | 13.370590 | 0.307008 | 4.43649 | 0.06 |
| 22.408610 | 223.833400 | 0.076752 | 3.96760 | 1.06 |
| 23.419320 | 10.663080 | 0.153504 | 3.79861 | 0.05 |
| 23.792870 | 25.059040 | 0.204672 | 3.73981 | 0.12 |
| 24.280210 | 24.563670 | 0.153504 | 3.66584 | 0.12 |
| 25.380830 | 84.704150 | 0.127920 | 3.50932 | 0.40 |
| 26.594250 | 4.398074 | 0.614016 | 3.35189 | 0.02 |
| 28.098140 | 1146.925000 | 0.076752 | 3.17581 | 5.45 |
| 29.862670 | 40.787540 | 0.127920 | 2.99206 | 0.19 |
| 33.452210 | 28.497950 | 0.153504 | 2.67875 | 0.14 |
| 33.873600 | 219.748600 | 0.153504 | 2.64639 | 1.05 |
| 39.717160 | 39.621590 | 0.127920 | 2.26947 | 0.19 |

Form P:

| | | | | |
|---|---|---|---|---|
| 3.529689 | 27.888510 | 0.038376 | 25.03247 | 1.18 |
| 3.662600 | 23.476020 | 0.076752 | 24.12437 | 0.99 |
| 4.400123 | 35.489730 | 0.038376 | 20.08230 | 1.50 |
| 5.382416 | 2372.071000 | 0.115128 | 16.41928 | 100.00 |
| 6.063667 | 11.157090 | 0.102336 | 14.57602 | 0.47 |
| 6.932938 | 221.108600 | 0.102336 | 12.75027 | 9.32 |
| 7.198101 | 32.781830 | 0.038376 | 12.28116 | 1.38 |
| 7.833931 | 49.410480 | 0.038376 | 11.28574 | 2.08 |
| 8.951140 | 142.715800 | 0.063960 | 9.87950 | 6.02 |
| 9.657990 | 31.260630 | 0.076752 | 9.15797 | 1.32 |
| 10.403560 | 56.011520 | 0.127920 | 8.50328 | 2.36 |
| 11.610730 | 160.530200 | 0.127920 | 7.62176 | 6.77 |
| 12.350530 | 86.125170 | 0.102336 | 7.16683 | 3.63 |
| 13.032840 | 36.368250 | 0.204672 | 6.79312 | 1.53 |
| 13.307100 | 30.614840 | 0.038376 | 6.65372 | 1.29 |
| 13.707020 | 29.934460 | 0.307008 | 6.46048 | 1.26 |
| 14.332740 | 22.274910 | 0.089544 | 6.17981 | 0.94 |
| 14.490940 | 17.291070 | 0.076752 | 6.11270 | 0.73 |
| 15.112550 | 19.244290 | 0.127920 | 5.86264 | 0.81 |
| 15.859560 | 90.625380 | 0.102336 | 5.58815 | 3.82 |
| 16.064370 | 58.597610 | 0.102336 | 5.51736 | 2.47 |
| 16.688640 | 238.859300 | 0.140712 | 5.31235 | 10.07 |
| 17.130230 | 54.009750 | 0.204672 | 5.17639 | 2.28 |
| 17.496240 | 35.200040 | 0.051168 | 5.06892 | 1.48 |
| 17.847610 | 129.373800 | 0.127920 | 4.96991 | 5.45 |
| 18.156050 | 23.965910 | 0.409344 | 4.88617 | 1.01 |
| 18.676350 | 51.434760 | 0.153504 | 4.75121 | 2.17 |
| 19.843090 | 129.746400 | 0.102336 | 4.47440 | 5.47 |
| 20.598120 | 42.555630 | 0.038376 | 4.31206 | 1.79 |
| 20.819570 | 121.577300 | 0.153504 | 4.26670 | 5.13 |
| 21.889910 | 143.646000 | 0.127920 | 4.06043 | 6.06 |
| 22.991970 | 85.011130 | 0.179088 | 3.86824 | 3.58 |
| 23.998440 | 65.841820 | 0.127920 | 3.70824 | 2.78 |
| 24.321900 | 58.779780 | 0.153504 | 3.65965 | 2.48 |
| 24.829690 | 53.545770 | 0.102336 | 3.58595 | 2.26 |
| 25.930430 | 46.740940 | 0.179088 | 3.43617 | 1.97 |
| 26.469120 | 13.342160 | 0.307008 | 3.36745 | 0.56 |
| 29.441520 | 13.563290 | 0.307008 | 3.03389 | 0.57 |
| 32.238140 | 10.968090 | 0.409344 | 2.77681 | 0.46 |
| 36.180110 | 6.661629 | 0.614016 | 2.48280 | 0.28 |

Form Q:

| | | | | |
|---|---|---|---|---|
| 3.260787 | 12.693460 | 0.102336 | 27.09616 | 1.11 |
| 5.608259 | 908.625200 | 0.115128 | 15.75857 | 79.68 |
| 8.367888 | 72.985470 | 0.179088 | 10.56676 | 6.40 |
| 10.418170 | 44.613940 | 0.153504 | 8.49139 | 3.91 |
| 11.246690 | 24.166920 | 0.153504 | 7.86763 | 2.12 |
| 12.344520 | 561.072400 | 0.115128 | 7.17031 | 49.20 |
| 12.777440 | 153.379900 | 0.102336 | 6.92832 | 13.45 |
| 13.576850 | 1140.351000 | 0.102336 | 6.52212 | 100.00 |
| 14.947770 | 188.392700 | 0.076752 | 5.92690 | 16.52 |
| 16.820880 | 820.353500 | 0.115128 | 5.27089 | 71.94 |
| 17.692630 | 73.823840 | 0.204672 | 5.01309 | 6.47 |
| 18.728990 | 157.793900 | 0.153504 | 4.73797 | 13.84 |
| 19.388890 | 217.951200 | 0.115128 | 4.57818 | 19.11 |
| 20.147680 | 41.924150 | 0.358176 | 4.40744 | 3.68 |
| 20.937470 | 159.415700 | 0.127920 | 4.24294 | 13.98 |
| 21.732220 | 221.688000 | 0.076752 | 4.08954 | 19.44 |
| 23.310170 | 134.005500 | 0.115128 | 3.81615 | 11.75 |
| 24.474700 | 52.413970 | 0.102336 | 3.63715 | 4.60 |
| 24.825740 | 48.549210 | 0.153504 | 3.58651 | 4.26 |
| 25.196770 | 14.342530 | 0.102336 | 3.53453 | 1.26 |
| 25.474120 | 12.290820 | 0.127920 | 3.49668 | 1.08 |
| 26.251990 | 115.046000 | 0.102336 | 3.39481 | 10.09 |
| 26.838030 | 16.341230 | 0.076752 | 3.32199 | 1.43 |
| 27.203510 | 32.787080 | 0.204672 | 3.27819 | 2.88 |
| 28.207540 | 94.352190 | 0.204672 | 3.16374 | 8.27 |
| 28.915720 | 23.455230 | 0.204672 | 3.08785 | 2.06 |
| 30.220390 | 39.440030 | 0.153504 | 2.95745 | 3.46 |
| 31.844520 | 50.706460 | 0.153504 | 2.81023 | 4.45 |
| 32.409930 | 25.599560 | 0.153504 | 2.76248 | 2.24 |
| 34.063480 | 8.783975 | 0.307008 | 2.63207 | 0.77 |
| 36.838030 | 9.840682 | 0.153504 | 2.43995 | 0.86 |
| 37.636080 | 12.850160 | 0.153504 | 2.39003 | 1.13 |

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

The invention claimed is:

1-65. (canceled)

66. A compound of Formula I: 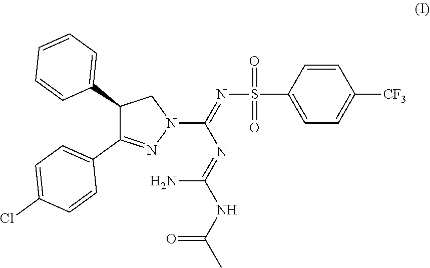 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]
which is crystalline and exhibits an X-ray powder diffraction (XRPD) pattern having characteristic peaks expressed in degrees 2θ (±0.2° 2θ) at 3.81, 5.86, 6.46, 7.46, 12.94, 14.50, 14.94, 15.43, 15.88, 17.17, 17.95, 19.44, 20.84, 21.53, 22.15, 23.34, 24.24, 25.11, 26.24, 27.38, 28.41, and 30.57, when measured using Cu K_{α} radiation.

67. The compound according to claim 66, where in the XRPD pattern has characteristic peaks expressed in degrees 2θ (±0.2° 2θ) at 5.86, 6.46, 15.88, 19.44 and when measured using Cu K_{α} radiation.

68. The compound according to claim 66, wherein the XRPD pattern has characteristic peaks expressed in degrees 2θ (±0.2° 2θ) at 5.86, 6.46, 7.46, 15.88, 19.44, 22.15 and 26.24, when measured using Cu K_{α} radiation.

69. The compound according to claim 66, wherein the XRPD pattern has characteristic peaks expressed in degrees 2θ (±0.2° 2θ) at 5.86, 6.46, 7.46, 9.85, 15.88, 19.07, 19.44, 22.15, 22.77, and 26.24, when measured using Cu K_{α} radiation.

70. The compound according to claim 66, which has a Differential Scanning calorimetry (DSC) thermogram that exhibits an endotherm having an onset of about 157.1° C. and a peak temperature of about 167.9° C.

71. A compound of Formula I: 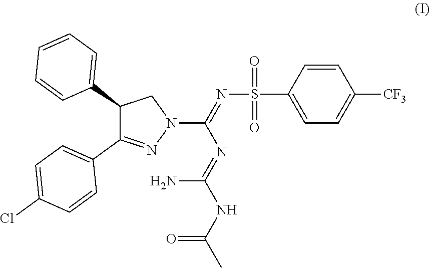 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]
which is crystalline and exhibits an X-ray powder diffraction (XRPD) pattern having characteristic peaks expressed in degrees 2θ (±0.2° 2θ) at 5.89, 9.39, 12.96, 16.96, 17.39, 17.80 and 19.85, when measured using Cu K_{α} radiation.

72. The compound according to claim 71, which is crystalline and exhibits an X-ray powder diffraction (XRPD) pattern having characteristic peaks expressed in degrees 2θ (±0.2° 2θ) at 5.89 and 17.39, when measured using Cu K_{α} radiation.

73. The compound according to claim 71, which has a Differential Scanning calorimetry (DSC) thermogram that exhibits an endotherm having an onset of about 152.2° C. and a peak temperature of about 162.3° C.

74. The compound according to claim 66, that comprises one crystalline form at a purity of 95% or higher.

75. The compound according to claim 74, wherein the purity is of 99% or higher.

76. The compound according to claim 75, wherein the purity is of 99.8% or higher.

77. The compound according to claim 71, that comprises one crystalline form at a purity of 95% or higher.

78. The compound according to claim 77, wherein the purity is of 99% or higher.

79. The compound according to claim 78, wherein the purity is of 99.8% or higher.

80. A pharmaceutical composition, comprising a compound according to claim 66 and a pharmaceutically acceptable carrier or excipient.

81. A pharmaceutical composition, comprising a compound according to claim 71 and a pharmaceutically acceptable carrier or excipient.

82. The pharmaceutical composition according to claim 80, that is formulated as an oral dosage form.

83. The pharmaceutical composition according to claim 82, wherein the oral dosage form is a tablet, a capsule, a lozenge, a pastille or a granule.

84. The pharmaceutical composition according to claim 81, that is formulated as an oral dosage form.

85. The pharmaceutical composition according to claim 84, wherein the oral dosage form is a tablet, a capsule, a lozenge, a pastille or a granule.

86. A method of treating a disease or disorder selected from the group consisting of: obesity, diabetes (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout comprising administering a compound according to claim 66 to a patient in need thereof.

87. The method according to claim 86, wherein the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, osteoarthritis, high cholesterol, and increased incidence of malignancies of the liver, ovaries, cervix, uterus, breasts, prostate, and gallbladder.

88. A method of treating a disease or disorder selected from the group consisting of: obesity, diabetes (type 1 or 2), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout comprising administering a compound according to claim 71 to a patient in need thereof.

89. The method according to claim 86, wherein the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, osteoarthritis, high cholesterol, and increased incidence of malignancies of the liver, ovaries, cervix, uterus, breasts, prostate, and gallbladder.

90. A process for preparing the compound according to claim 66, comprising:
suspending a first crystalline form of Formula I in water, the first crystalline form exhibiting an X-ray powder diffraction (XRPD) pattern having characteristic peaks expressed in degrees 2θ (±0.2° 2θ) at 5.89 and 17.39;
subjecting the suspension to temperature cycling, between a first temperature and a second temperature lower than the first temperature; and
retrieving the compound of Formula I as a second crystalline form exhibiting an XRPD pattern having characteristic peaks expressed in degrees 2θ (±0.2° 2θ) at 6.46, 15.88, 19.44 and 5.86;
wherein the first temperature is between 45° C. and 65° C. or between 50° C. and 60° C.; and
wherein the second temperature is between 5° C. and 35° C., or between 5° C. and 25° C.

91. The process according to claim 90, wherein the first temperature is of about 60° C. and the second temperature is of about 25° C.

92. The process according to claim 86, further comprising between the temperature cycling and the retrieval of the second crystalline form: stirring and maintaining the suspension at the second temperature for about 1 hour to about 12 hours, or wherein stirring and maintaining the suspension at the second temperature is performed for about 3 hours to about 6 hours.

93. The process according to a claim 86, wherein retrieving the compound of Formula I as the second crystalline form comprising filtering the suspension.

94. A process for preparing the compound according to claim 66, comprising:
solubilizing a compound of Formula I as a crystalline form exhibiting an X-ray powder diffraction (XRPD) pattern having characteristic peaks expressed in degrees 2θ (±0.2° 2θ) at 5.89 and 17.39 (when measured using Cu K_{α} radiation), in a solvent selected from the group consisting of methanol, ethanol, n-propanol and isopropanol, to obtain a solution;
adding water to the solution until a solid precipitates; and
retrieving the compound of Formula I as a crystalline form exhibiting an XRPD pattern having characteristic peaks expressed in degrees 2θ (±0.2° 2θ) at 6.46, 15.88, 19.44 and 5.86;
wherein solubilizing the compound of Formula I in the solvent is performed at a first temperature between about 30° C. and a boiling point of the solvent or at a first temperature between about 35° C. and about 60° C.; and
wherein adding water is performed at the first temperature.

95. The process according to claim 94, further comprising cooling the solvent/water mixture to a second temperature that is lower than the first temperature, wherein the second temperature is between about 5° C. and about 30° C. or wherein the second temperature is between about 20° C. and about 30° C.

96. The process according to claim 94, wherein the solvent: water ratio (by volume) is between about 1:1 and about 3:1, or wherein the solvent: water ratio is between about 2:1 and about 3:1.

97. A process for preparing the compound according to claim 66, comprising:
providing a slurry of a first crystalline form of Formula I in a solvent selected from the group consisting of water, n-butanol and an MIBK/n-heptane mixture, the first crystalline form exhibiting an X-ray powder diffraction (XRPD) pattern having characteristic peaks expressed in degrees 2θ (±0.2° 2θ) at 5.89 and 17.39;
stirring the slurry at a temperature between about 50° C. and about 70° C., or at a temperature is between about 55° C. and about 65° C.; and
retrieving the compound of Formula I as a crystalline form exhibiting an XRPD pattern having characteristic peaks expressed in degrees 2θ (±0.2° 2θ) at 6.46, 15.88, 19.44 and 5.86.

98. The process according to claim 97, wherein the solvent is water or a 1:4 (v/v) MIBK:n-heptane mixture.

99. The process according to claim 97, further comprising adding a seed of the compound as defined in claim 1, to the mixture.
